SEQUENCE LISTING (128 sequences)

SEQ ID NO: 1 - DNA, 200 bp, Homo sapiens
  misc_feature (66)..(66): n is T or A
gagtgcttggtgagtgggaggaagatgctggccatggggcccagggcggggagccccttggcaccncgggaaccccagcccaggaggtttcactggaagagaggctgggcttgagtgagaagtgagacacacgcgagtttccggtgaactcggcaccaaacacctcagtttgctgctcagatgaggtgtcagaaaatgct

SEQ ID NO: 2 - DNA, 107 bp, Homo sapiens
  misc_feature (72)..(72): n is G or A
ggggctgcacagcagggagagtgctgtgttatgcgaggaggttggagaaatcctccccatgagataagatgngaacagagatcgggacgaaacaaggggaggggaca

SEQ ID NO: 3 - DNA, 22845 bp, Homo sapiens
ctcagagcctcatctgaggtaaaggagcaaagttgggattggggtcaaaattcactttaactccaaagcccacacacttaaccaccctgcctatttctgtccaaatgtcacctgtcctgaatggagtttttccccctgtacaactgtcatcaacctgttcgggccctctcactgacaggcaggtccctacctatatttgaggggcagcccattgcatttctggacagctctcgccattagggtgcacacacgcaccacctctgtgaacagggctctggctaggccactcctcagcagctcttgttgcttccccatggccctggtcagcagctggagtgcagagaccagcgggccttaccaagccacagctccaggccatgcccgtcagcaacacttttcactgtgactctctgggaggtgcccagggcagagggtgactccaggatgggatgcctttgcagtgggtgatggtcttcaagaaccagtctcaaacttgtagtgacaggcaggtgccctccctggggagcccagagccctatgggaacaccacagtgatgctcagaagtccctgaaggcagccgccctcgactgctcatggctgtgtcattcacgcaacagcagaggacgtggcagggagttcgccagggggctgatgatggtggccagctactccagaaccactcactttccccagggagaggcgacggaagaaggcaaacctgacagtgcagggggcaggggacacttggcctagaggagagaagactgtgggggtgaagagggtgatggtgcaagagctgggtctccagcaggctgtcccgggcaggagacaggaccggctctctggtgccaagcctattaagtcctgccacgtttgctgaggaaatacctgcgggggaccaccccacagcagacacctgggctagtgacaaccaagacaccacatgcactcaagatttagcccctgaaagaactctgtagaaacaacagagggtcgggttgggggagctgtatgtcctccctcgggagggcctcccctctggaggtgagcagtggctgcttccgcaaaggacgagagagtcttggaagccaggggaggtgggtgggagtcagggtctcggcattcttctccatccagagcctagggtttaaggctggaaggatatttacggagggtaatgactttgtaaacaagacctagaaatctatctcccagtagcatcatctttgacccatgtcctctctgcccagccaaagtattgccctgcctatccagaaagatggaaacccttccagtgaaattccacgtgactccctcaagaacaaagactggaattgaggctgatgtggggcaaggttcctcgggatcctccaaggccgtgctctgagccacacattcaggacagcctgcgaggcaggtgcacagtgcggatggccgtcccgggccccactaggggtctgcagctcgccacttcctcagccaccgccgagctcctgtccctcacaccttccaggctgctccccaggaagggggacctgtggtcactggcagtgtgccctgggctggagagggcgtggggctctccagggatgagtgacaccttactgagccacagaaccgagctgcagctggtggcgacagggaaggggccctgtcaggaatgagaaaggaagacagaacaaggtaggtaagtgtcagcactgcaactgtggctgggaacctccctccctggggactgagccctggtggtgtttgctgggagttttctctaaccacgtggcctaagttctttcataaacaaggcaagccctcagcaggactcagagagaagggggttgagaagggagaagagtgcttggtgagtgggaggaagatgctggccatggggcccagggcggggagccccttggcacctcgggaaccccagcccaggaggtttcactggaagagaggctgggcttgagtgagaagtgagacacacgcgagtttccggtgaactcggcaccaaacacctcagtttgctgctcagatgaggtgtcagaaaatgctcccagtcacttggcaggtttctccgaggctggccccctgtcctgcgcaggacgagaaggagcaggccccaaggggaccaggcccgcatcttatttccttgtgaccgaggcagcagcttgaatgaagcctggccttcacctggcccagcggcttatcgctttttcattagggtcaaagagaaatgtcttctgcctatgcccttcatctagctggtgcattgtaacacactccaaacctgtttcccacttcgcctgcctcccttctaagaaggtccggctggggccaggctgactatgttccagacaggacagcctggccagggacacactggccagctcctatctggcctttctaggggtccaagctggggtcaggactggtggctgtctcctcagctaccctcccccacactcataaagccccattatcttcattcatttctcagctccacccaaaagcaagagggcaagggtttgcaacaaactttcccagatcaactgatttctcggcagcaagggagggccccatgacaaagccatttgaaatcccagaagcaattttctacttacgacctcactttctgttgctgtctctcccttcccctctgctataccttttcttggggaaagtgattctctcacagcgtcgttcagaaacacccccagccatgtgctgcctgcctgggcagacgctgctgtgtggtcagaggtgacgcaggcaccaaaattaaatatagcaactctcgtcagctccactctctgcatcactgtggacaactgtgcgggggctggacaccatttccccaaaccctcaagggaggaaggcccaagccaccgatggcatcccctgtagacccacagcccgatggcctgttggccacacagtccacaactccctgcccagcccagggccctgtgccagaggagctgtggtgatgccacattggaaaaggcaatttttatttactcttctttcttctgatctgtttcccacattttctataatgagcatgcattcattttccaatgggagacaaggataaatttcatttttaaaaagcaaaaagaacatgaacgtgatgtctgctccatggaggatgtggaccgagacaggccagtgaggagacctggttgcagcatcccccggctggaccagcaccagcacccctcagaccagcctaagtcctccctggggagccggccctctcgggagaggaggtgggggtctgcaggggcctgcaaggcagcccctcagccagacaggactagcaccgctcctcccatgggttgggcactcttggacccatcagatcccacattgccaaagacagaggaaggtgctgccttcttgggggtcttcactaggccctggcctgccagggcacaggagccagcgggagtgggaagggaaagggtaggccgcccatctagctctgaggctgaagtgacattaacactatggggacagatggagcattcctgcagactcctcagcagctctgcctctcttgggagaaggaccttccacctgcttctgaaaggcaagagaagccaacagcctgcagagctacttccccacagctccgcaaagcggagtctatgccactgcctgccagcctcgtttctctcctcccctcggtccctgtgcccttggatggggccctccatgtgtctgtgctctgggccccatcccctcccctctcaggaacacaggcagagcatatagccgggggctcagggtgaaactagtcccagcttcaagccccagctgtgtccctgacttctagtgttgttacttcagccacgtctcagtggctccgtggaaatggagctccactcattcattcaacaagtatggattgaggccttgcctgggtgccagaccctgctctaggtgctgggggtccactaaacaaaacaaggttgcttctagcagggagacagaataagccaacgagagaacactcagtgtgtcgggcggtggtagccgctaccgtgggaaatagaacagtccggggctgcacagcagggagagtgctgtgttatgcgaggaggttggagaaatcctccccatgagataagatgggaacagagatcgggacgaaacaaggggaggggacacccatgcacagatcggggcaaagtcatcaagggaaagggaactgcaggtgctaaggtcttgagcaagagcgagctggggatgcccttcccagcactcacgggtggccctcgggctggagcagaggccacggcaagggcagcaggagcccagcagggcctcagaggccaggaccgggcttcattctaagtggaatggagtgatgtggtctgactcacattttaacaaagtcaccgtggctgctgtgtggagaacgcaacgcagggcgaaggcggagcagggagatgagcgccaaggtgcctgcagagatgctggtggggagcgatggtgcttgtggtagtggaggactggagaggggcgtgtgtgtaagatgcatgtatacattatactgctatcgtatacattatttacttaaatagtatattatataatccataaagtatagatataaatatataatatataaatgtgcatatatgttaatatttacatttgctatgcaattacaattttaaaatcttctattacagaaaatttcaaatatacacgaaagtcactcccaagcatccattgcccagcgtcaacaatgagcaactcatggccagttttgtcaattttgttccatctctacccccgccccttccccaccaccaccaatccgagatgtcacatcagtgaaaggagaggctttggggtggcttggatatgagcgtgtgaggtcttcccgtgttgtgtgtgaacaactgcaaggatgaagctgccgctcgctgagctggggcagcctgtgcttgattgtgtcccattcagaacacagctgacaacctacgacgcagcctgcagcccaacacagctgccctcccagatgcccatactgagttccacctcatctggccacttcctgtgcccccaactgcactcagatatctcatgaggtcactgattatcaactccactccgtgcagcaacgcatgtctgcccttatctagcctcacctacgaggaacattgctgccgctgaccactgctcctcctccctgggcctctctcatgcctggactctgcaccctgcctcaccaccttccagtgtcgcaggagtgccctggctcatttcttgtcccctgtcttctctcaccccttggccatattgttaggtcaacaggagtggaggagagccagggtgacaccattttaaaatcaactccatcttaaaactagctaggcatattccttgccagtcactacccatggtcatgagatgtttacgccaaggaagcagtttagtcacgcccgcaaggacaaactcctacaacagcagaatgtctgggtgtcctgatgtctcataacaatatatgctttcaagctatttacagtaatgctttgatgtacttactcactaaaaggccaaggataaaacattatttaaatcaacaaagtactaaattttgtgacactgtcagcccacctgcacatagacataatgtagcttggcttttgtatagacaagacccctatataagaaaagctttaagcagacaaggcgttcctgctcatgctttctgaggacaccctactctgagtagttttctacatcctttctttaccacacaccacgactcaccttgaattccttcctgtgcgagatccaagaactctctcggggtctgaatcgggacccctttttccagcaacactgtgttcttccccttggcttagtcaccctccatgtctactgcctgcaggctggaccctcccctgttctcagagtatggcccgtagcagcccctcagctggtggctcccacccgccgcctgccctctcccctgcctcctccccaagccagagacctggggccacctgcgacttcctgctctccctcacccccacattcagtcacttccaagtgctattaatgatgatggtggtggtgatggtgattgctggcattttccgggggcttgcaatatgccggtcaccacaccaaacacttcatatttgtcaccttctatagtattccctcgcccctgtaatcagcccagttttacagagaagcctggatgggggaacttttccaaggtcacccagagctgggattcagaaccagtcaggtcccgtgggcaccaagacaccaggtcttaacctagacactgtgctgggccaaatgactcaagagcccacagctctccctgccaccatcccacccgattcgcgccagctgcctctgtcacgggcgcctgcaatgctctggctgggtcccctcggcccgtgctcccctcctgggagccctctcttcagtgacctcggggccttccacctgctgtcccttgcccgcactcaccccaccctgcagcccctgctcctggctaaagccggctcatccctaactctgcttaagtgcccctccctcagagaagtctcacctttttccatgactaagcctgtgggggttggaaagcactctcctgggtgctggcctgcaggactgacagaagaggagggaggtgagattcacccgactcggaccacaggaatggctgggacagcaagcatcaatgaacgaggcccgtggagactgggctgcattgtgcgacctgtattcctttctcctagttgactgccgcgtttctgactcctttgaagcgagcatctggcttttccaattagatgaaggctgacaagctgtggaggggagggcggcagataccatgtacctggtcattcagactaggggtgtccttgagcagactcatggtgtggaagtcagaccgggagtctcctggagcagactcacagtgtagggggtcagcagaggcagcagctttggaatcccggcactgcagcctcaggggtggctcgctgagtgggtcaggtctttagggttctgggcccagcctggagcctgcccctccagccctcctgacattcttagaagcacctactttcctgcatgaaatcctttcctgactaaagcacccacagctgtgtctgttccctgtaatgaatccagatactaaagtaggcgggctgcagtgtggagaccgtgacccaccagaaacaaggacggcaactcaaagacggaggaggcacatccaggaggaacctgtggggagggcccgtctggccagatctccactgccctgtccagacttgggcttgcctaatagatgaagcatcagtcatttcagcaactcaagataggagtcatcattatcatcatcacactcactgtgtgccaggcactattctaaatacttgaaaactttaaatgtattcattcctcagagcaacttcatgagacagggacagctatgacccctatttcacagatgaggctgagtagcgtgcccaaggtcacacagccaggaggcacagcagccaggcctgacagaccacctgggcccagcgtccgctctcttagccaccgtgtactatagcagcctctgttaacagaccccctttctggatgacacatgccaagtactttccatggaaccactcacttgctcctcacaaggaagagccacattattcccatttcacaggtgagaaaatcgagacccagagagagttaatgatctactcatggtcacagagttgataagggctcatttgctggactcccaaacgcagtgctcataactgctacgttccagggcctgaaggaaaaactctgcatccatggaggggccggcgctggttctcagctctcacacaggggaggggaaggggcctgtgaccgacacagccagagacagcagtattcaacctccctcctgaactttggtgtcaggcccaccacaccccgccaaggcactgcccatggccctgcaggctcggagactccttcgcagtggtggtagtggtggtgatcactgccctcctctttgtccctgacaatgcaggcacccaccttccccatctctacccacctgccgcacctgcagctgccatggtgctgtccctgcaggcgaggatggcccatcccccacttctgccctctggggagactcctggtcactctcgaatgttctggacagtttatcctttcatctttggcctcatttcaccattgaaacaaacaaaacaagctggattctgcttctgagctgaaggtgcccacctaatattcccttttcactcaccagctctgccctcagagcctcaagcccagggtctgccctttagtgggtgcttagaaaaacaccagatggacacataaatggctgttccactgcccccacagacgccccagaaccccgccctccccaccagctccccttctgcatccccgactctccttgagaacctatttggcagaagctctccacccagcaagtccgcagcttgatgagctccctcctgtgttaactggaaccgctgctgtacttcattccacataatagttcatcggatccaaagtccccacctgctttggaagcaaccacctgctcttctcataactctcctccagtttgtgcagtgaagaatcaacctttatccaagaagtctggcctttgtcctggctcttgggaggtcctaccagctacaaacccttggagtaaacaacgtgcctagtccttgtcaccagttcccaggaggtagccccaaattcctagggatttcccaagtgataggagtatcttattattcatggtggtctctgagagtttatgcgagtgaagtgactcatggtgggccctaggtagtttttgctgacaatacgaccatggaggggctggccacgccactgaggttctgtgatatcagcctggcctcccggaaggagaccaggaagatgagttcaacccagtggccaatgagtccatcaaccacacctatatgataagactcaaataaaaactctggaccccaaagctcaagtgagcctccctgcttagaaatagtcagcattttgtcacacgtcaaagtgctgagaaggtgatgcctctgacgccacacggggaagacaatgagagctttgtgtttgggcccctcctccatctcgcccctgtgtctcctcttttggctggttctgatttgaatgcttttgttatgataaaactgtggccttacgtatagcactctcctgagttctgtcagtcatcctagtgcattctggaacctgaggggtagtggaaactcccagatttgcagccagtcagcagtgagggtgggctgggaacccctgaatgtgcagctggcgtctgaagcaagggcagtgttgtgggggaccatacccctcacctgtgaggtgtggctcatctcaggtggtttggcatctgaagccactgcatttagtttggtaaccttgctgcccagtcccaatggaaggatcctaaatatggtctaaggacctcctgatacaattatccagattctctccttcacagaacttgaggcactgcgataagatccaaaactatatatacacagtggaatactatacagccttaaaaaagaaggaaacgctgtcattcaccacaacatggatgaacctggaggacattatgctaagtgaaaaaatccaggcacagacagacaaataccacatgatctcacgcatatgtgaaatataaaaaagccaaactcagggaggcagagagtaggatgatggtcaccaggggctgggagggtggtgatcaggaagatgttggtcaaaggatataaaatttcaactgggaggagttaagttaaagagagccattgtacgacatggtgacaacagttgatatcaatgtcttgtatacttaaaaatcatgaagacaggccaggcgcagtggctcacacctgtaatcccagcactttgcaggggctgaggtgggtagatcacctgaggtcaggagttcgagaccagcctggccaacatggtaaaaccctgtctctattaaaaatacaaaaattagctgggcgtggtggcaggcacctgtaatcccagctactcgggaggctgaggcaggagaattgcttgagctcaggaggcagaggttgcagtgagctgagactgcgccattgcactccagtctgggcaacagagcaagactctatctcaaaataaataaataaatcacagagtagattttaaatgttcttaccaaaaataaatatgtgaagtattgtataagtagcttgatgtagcaattccataacatgcacatttcaaaacattatatcatacagcacaaatatgtgcaatacttatttgtcaatttaataataataataataagggaagaaaagatccaaaacagaggcaaaaccttggccgggcatggtggttcacgcctataatcccagcactttgggaggctgaggtgggtggatcattttgaggccaggagttcgacaccagcctagccaacatggtgaaaccccatctctactaaaaaaaaaaaaaaaatacagaaattagccaggcgtggtggcatgtgcctgtaatcccagctactcgggaagctgaggctggagaatgccttgagcccaggagatcaaggctacagtaagctatgatcaccactgcactccagcctgggtgacagagtatgggggcagggggtggtgaggggggggcggggaagtggaacagaggcaaaaccttagcaacacacatttttagatgatcttccagaataaattcatagggaggcccaggcacagtggctcacgcctgtaatcccagcactttgggaggccgaggcaggcggatcacgaggtcaggagatggagaccatcctggctaacacggtgaaaccccgtctctactaaaaatacaaaaaattagccgggcgtggtggcaggtgcctgtagtcccagctactcggggaggctgaggcaggagaacggcatgaacccaggaggcggagcttgcagtgaactaagatcacgccactgcactccagcctgggtgacagagcaagattccatctcaaaaaaaaaaaaaaaaaaaagaaattcatagggaaaagaaggtcagagaccaagggaagggaaggttctgggagaaaagcagggggcaggcagggcccaagaatcctgctgcccatgagcccttactgggaggtggggtggccttgcacagggcccaggcacctgagtgagtggtggggtccttacgttcactgctggggtgaggcaatgagcaccttattgtgtccacatgaattcaataaaaaacaagcagggcgggtggtggggcactgactaggagggctgatttgtaagttggtaagactgtagctctttttcctaattagctgaggatgtgtttaggttccattcaaaaagtgggcattcctggccaggcatggtggctcacacctgtaatctcagagctttgggagactgaggtaggaggatcacttgagcccaggaatttgagatgagcctaggcaacatagtgagactcttatctctatcaaaaaataaaaataaaaatgagccaggcatggtgcggtggcacgcacctactgctaggggggctgaggtgggaggatcacttgagcctgggaggttgaggctgcagtgatccctgatcacaacattgcatttcagcctgggtgacagagtgagaccctgtctcagaaaaaaaaaaaaaaaagtcattcctgaaacctcagaatagacctaccttgccaagggcttccttatgggtaaggaccttatggacctgctgggacccaaactaggcctcacctgatacgacctgtccttctcaaaacacctaaacttgggagaacattgtcccccagtgctggggtaggagagtctgcctgttattctgcctctatgcagagaaggagccccagatcagcttttccatgacaggacagtttccaagatgccacctgtacttggaagaagccaggttaaaatacttttcaagtaaaactttcttgatattactctatctttccccaggaggactgcattacaacaaattcggacacctgtggcctctcccttctatgcaaagcaaaaagccagcagcagccccaagctgataagattaatctaaagagcaaattatggtgtaatttcctatgctgaaactttgtagttaattttttaaaaaggtttcattttcctattggtctgatttcacaggaacattttacctgtttgtgaggcattttttctcctggaagagaggtgctgattggccccaagtgactgacaatctggtgtaacgaaaatttccaatgtaaactcattttccctcggtttcagcaattttaaatctatatatagagatatctttgtcagcattgcatcgttagcttctcctgataaactaattgcctcacattgtcactgcaaatcgacacctattaatgggtctcacctcccaactgcttccccctctgttcttcctgctagcatgtgccggcaactttgtccacggacacaagtgcgatatcaccttacaggagatcatcaaaactttgaacagcctcacagagcagaaggtgagtacctatctggcaccatctctccagatgttctggtgatgctctcagtatttctaggcatgaaaacgttaacagctgctagagaagttggaactggtggttggtggcagtccagggcacacagcgaggcttctcccctgccactcttttttctgagggtttgtaggaagtttcctcagttggagggagtgagagctgctcatcaaggacttctctgtccggttggaggttaactctgtctcttgctctctcatttctgcctggaccaagactctgtgcaccgagttgaccgtaacagacatctttgctgcctccaaggtaagaagccgtcccacggtctgttttagcaaatggggagatccatccccaaatgtctgaacaagaaacttgtctaatggaaaacgagcgggcccaaattaactctaaggtgttagatgttttcaaagaacgagaagtctgatctttactcttaagcatgttttggtctttctggtttcacttgatttagaagacatgtaatagaaagcttacatgctgtagtcctgactcagatcctggtcaaagaaaagccctcttgggttttacttagctttggcatagtgcctggaacgtaggaggcactcaataaatgcctgttgaatgagagaatttttctggcccatacatttctgaaaaaccaaatactctcacagaaacagatattgagatgacaggttgagggagctttcattttgtctaagagacttcctatggcaacagaaaaggtatcgccagagcccctcctcttccacagcctggccacctaacagccctctgggttccggggctggccgtccagagctcctcagcttgctctggccggccgaactcccctccagctcggtctggaaccatcctgctgggcagcgtccagcacatccctgcttcgggctgcctgggcacctcgcctctctgcctcctgtgctgcctcacccccacccctctatctgtagtgggaggagatagatttgacagctgatagtgcattttctctgacaaacacatgactacagccgtatcaatagttttgtgcatttcagttcctgttttcatggaaacacacggctgagaatgaaagccccaaagcctcaatttcacagtggtctcctaactacctgctttccatgcaaactagggagatgatatggccaggagtgaagccctgtgtgttgggcagggtcacactccagcacccagaccatagaacagggcccatcctgcttcatgagggaaactgctcttcgggcctttagctggactatctcatttcattagttatcccgggagtccgatacaggatgagattctgaagggcaaatacacacttttttttttttttgagatagggtcttgttctgtcacccaggctggagtgcagtggtgcgatttcagctcatagcagcctccacctcccaggctcaagctatcttcctacctcagcctcccaagtagccgggacgacaggtgtgcaccaccacgcctggctaatttttgtatttttttgtagagatggagtcttgccattttgcccaggcttgtctcgaacttctgggctcaagcaatccgtccacctcggcctctcaaagtgctgggattagccactgcacctgggcaacagtttatgtgtgtgtgtgtgtgtgtgtgtgtgtgtgtgtatatatgtgtgtgtgtatatatatgtgtatgtatatatatgtgtatatatatatgtgtgtgtgtgtgtgtgtgtgtgtgtgtgtgtgtgtgtgtgtgtgtgtgtgtgtataaaatctccaagtccatccaaccgagatggctcctactagaagccaagagtccaccgggttgagcactgggtctctggaggcctgtggcactgctgagaaggctctaacaaagccaagggaagggccacctcactagaagccaggcctggaggaagggtgagggctgagggcctggaggtaagactgcctgtggttttagacccagctctgccactgactagctgtgtggctggccttcagcacatcttcacacctctctgcacctcagtttccacatgtgaagatatgaaagtgattctgaaggtgattgcaaggttgattggaatccagctcttgagttagtgcaaagtgttattgtgagatgatataaccacgattaaaagcaagaacaggtgcagagaagcgatgattctaagaaggaggggaccgggttggaaaggatcaaaccatccaggatgccgagtctggggcaatccatctgggctgtttctggaagacccccgggtgcaggccaggacactgctgccctcccgtccttaactcccctcttcactcagtcctcactcacctccctctcacacacacaaacatctcctagaataatccccactgcctgccttcactcttacccgtctcatttgcctcccctgaacttcatcctcctggagttcacgatctcactcttcactcttttcttcccctcgaagattcagcactgcttacttacatgttaagatatttcagaacagtgaaatgttgctattttcaaaaacctacaaaggtggtatgcagaggaaaaggtacttctttgtgttcccaaagaaaacatctttccaaaatccagcctattgattttatttcttcgggggaacaagaattttagtatctctaagttgggtagcattctactcttggcagttgctggaaagaaggcactggtctaggtcctgggcttcacaggtaacacctgtcagggtgtctatgaagtcaaggctgtctgaggaacagcaaagtgggaagaagcaagctggctggctgatgaagggtttcttgggtggacaagtagttggagctatttcctatttaccaaagagagctaaagttcataattctacagagagttccataatgaacctcaaatacctctgttttttgaaggagtttctcatatacagcactagctgactatcctgggcaggatgggagataatgaatgcagtgccaatcgggctggatttatatggtcctcagtgaggctggtcaagaaccgagttagaactctcacagagtcactgccacagaagaaatctcccaagtggctgtttcctgacattcccgggagggacaggcctccttctgagtcactccctaagcagttctgaactgtgaggtcagccaggctgtccaagtgcactccctgagccactggcagacacactcagcagccagagctagacaggcaggtggtaggagtccagggccacggcagggatggagtgtcgccccctcgctgcgataccagagcaactaaaacgttaaggccttgcactaaagctgcccttaggatgcattcttttaaagtttttccatttaatgcagactcttttcaattcttattttatccttgtttcctttagaaagtcctttgaaaaatatctttagagggttttttcctatactatgtggccatatacgggtcaaaattaagtttaatttccaggctccaagccagcgtttcagaaaaatctcaccaaggtttgtggtaaaagaagcaaagggctgactttttggttttcttgaatctcactgttccctctgcagcagcatgcatgtctgcccacctccagacacacaggcaccatctgccgccccccatcagcccgtgtcccttccacctcgactcgcctacaaagcccagagaggtctgtttcttggcccccagagcccaaagatactgacacactcttacatttccaactagaatcaggaacgaggagtgactctcagtcagttcattaagtaaatgtctttctaaccgctctgcccatgggacatcacgccccacaggggaaaggggaagcttctgtagcctgggattctggtgcctcagtctgggtctagactttcctgaaaaaacgttaaaatatgaactgcattcctagaatttagcctacataaataagagatgaacacaaagatttctatagtttactcactgccgcttatttacagaagcaaaaatctgccacgataggggcctgacaaatgacagtaccactgtgcaatgcgtttctacgcagctctcaatcccatgttctctaataccaccgaagggcttaggaaatgcttatggtatatgtaaagagtaaagaagttacaaaacagtatcaacagttgacccctattttaaaaagtatttttaaaagtgtgacgatatttaccaaaatattaacagcaatagttacctctggctggtgggatgagtgaatgtatttttgttgaatatatgttacctttatagtaaatatatgttatcttgatcatcagaaaaaaaaatatgtaagaacttgaaagctgcttggacagcgctgctgatagaaacccctgagcatcttgtcactgttcttctgattcagagggtctgggtggggcaggggtggtctgagattctgtatttctaagaagctcccagtgatgtccatgctgctggtccatggaccacactttgagtatcaagggaccagagcatgtcgggggagaggctggggatagctttctttatctgaactggataaaggaactgggctcaagctaagaaccctctccaggttctgcatctttgttcttcagtgaaaaatgagaggacacaccaggccaggttcagactgagacacaatccctctcctgggttcccaatgacttgtctcttgtccattcccttctctaaggctaagggcccccaggaagagccatgtggccagaccctcacagttgctggcattccaaggagattctcactccgcatcatttggggccaaaaggccccttacagaagctctgcccaaggctcagatcaatggcacctgctcccagagcctcctctgatctcccaggacacctttccctgatctgtgcacttatctcttgctgcctggcaaaatgtcttagctcctcacttgggccatgtgctgctctcctctcccatggggagagccacacggagagtgctggccaaagcagcagagttcaggccaaaggatgtgcactcatttattcaacaggcatgcaggatttccagggaaagctggattttaaaacctctgggaacaagagcagaacctgactgagagctcatgtgggcacttttcatagcagaatagctcatgaggtatagagacacggacgcagaacgtgggctgtagcgacagatggtcctgcattctagtccccactgtgccttttcctcatgggatgactttattcaggtaccctttcggcaaaatcctccaagagaaaggaaactgggaggttctggggagaaggctgctgcgtttgcaattgggagaggttgttgacagaggtttatgtctgtggcaagcagccttccttcagtggaatacttgaagacaggtctgtagttgagcaaactcacctccatttgtcctcctggaaagaagaaatcaagaggaaaaatctctctcccatcctccaaatggagctggcacattgctatctgtggcatttgtctttccagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttggtaagctgcactgtattcctggcaagccggccgcgtggctcctggtggacagcagcctcacttctaaacactccttaggagctgcagcacccttggtcaacccattcattcattcactcattcaataagtatttgctgaagttccacaagtgctgggtgtggttctaggtgctgaggacgtgtcactaaagacacgcaggccgagtccctgttctcatggaatgttctaatgggagagttagaaaaacaaacatgtaaaatgatggccagcagtgatacgtgctacaaagaaaaacatagaaataaagaacataagagtcatgggggagggggctgacttaggagctggtgacattatctgagcagatatttgaattgagggagcaggccacatgactaactagggagaccattccaggcagaaggaggaggtatgcaaaggccttaggatggaaatgaactaacttcctgtatttaaagaccagtaggaaggccagtgtggctggatcagagtgagtgaggggtagtttccaggacagcagatcacacaaggcctttagattccaccacgagtatggcagggaacacctgcagagctttgggcaggacaaagactgtacaatctgatttacgtgatttaaaagggtcagtctggctactgtgtggtaaataggctgaaagggggaaagcatagaagcaagatggcctgttgggaggctaccacagtaaaccaggctagagatgatggtggcgtggacagaatgaagcaagatggcctgttgggaggctaccacagtaaaccaggctagagatgatggtggcgtggacagaatgaagcaagatggcctgttgggaggctaccacagtaaaccaggctagagatgatggtggcgtggacagaatgggagcagttgaggtgaacagatttgggatatgactaaaaataaaaccagaaggatttgctgacagatcggttgtagggggtaagatacaggggaggaaaagatgacctctttgttcctgcccaaacccctctggcgatggtcagtactgtttacagagagatgaaagactggcggcaaggcagggctggaggttcagcagaagatcaagagttcaattttgtacatcgtacatgtaaggtggctcttggatagccaagtgaaggtgttgagaagatggttagaaaagtctggaacttaggggagaggtcagaacttgcaatacaaaaaggagagtccttagatagatactgctgaaaatctgaatgacagaaagggagagatcaaaggactgagcctgagatcaacacatggaggtcaggagaggaggatccagccaaggggcctgaggaggagtgaccagtgaggcaggagaacactggagagtgggcggtaccccaggaagccggtgaggacactcaaggagggagggttgactgtgtcaaatgtactgaaaggacaggtcaggtgaggaccaagaaaggcccctgggtttggctgatggaggccatgggtgaggctgatgtaaatggagaggcaggaaggaaagcccagctggagtgggctcaccgaggatagggtggcgagaggagacaaagaaggaacagtgagggcagaacactctttgaagatgtttagctataaggctgcagagaaactgacccacagctgcagggtggttatggagtgagggaagctcttttaaggttgggggtatacccagcatgttaatgcacctgggggaatggtccagtggagcaggaagaactgaagagagcagaaagaggaagaatcattagggggcagaagtccttgtagcccagagtggatgttatctaatatcgagtggaggaattaattggctttagaggagaacaaggacatgtatcccctctctgggcctatcaccttgtagacaatgggataggtcatgggataggaacttggcacaacacatgttctctcttttaattctctccattatcttatgaagcaggcaagtaggcaaacaattgtcccaactttacaaaagaaactgaagcttttataaattaagtagtacatcctaagcaatacaattaataaatggtagagctgagattcaaactgaagcagtggcctgggggtagcatctggaatccttcccacctttagggctgctgtgctgcggtgctgctgtttaatggcacaggagggccacatgactgaatctctctcagcagtccaggcagtcatgcagaaggcccagtagagcaccgggcaggtctgagccagcatcttcaagttccaccctctgagcaagcacctagctgtgacacacctctccagagactgcactcccccccgcgccacccaccccaaaagcagataggtaatggtatacagtaaccatttctagaagtgtaagtagtatgcacccaaaataggcaaaacctgctggcctagtgatagagacaactcccagtcaggctagactggaggccttggttttataagtgttcaggtgacaagtgccacagtaggcttgatcaagtagacaggcaggcaagacaaatgcttaccaatgcaagctaatgaaatgtttcttttgcagaattcctgtcctgtgaaggaagccaaccagagtacgttggaaaacttcttggaaaggctaaagacgatcatgagagagaaatattcaaagtgttcgagctgaatattttaatttatgagtttttgatagctttattttttaagtatttatatatttataactcatcataaaataaagtatatatagaatctaacagcaatggcatttaatgtattggctatgtttacttgacaaatgaaattatggtttgcaacttttagggaaatcaatttagtttaccaagagactataaatgctatgggagcaaaacaggaaagaccacttccccctcgaggggttccctctcgagttagggacataacacacaagataattaaagaacacaaggccatacaagatgtaaataagacaccttgggtccaagagtgctatggattaccatatatttctgaacagaaaaggtatcaactggatgtctaaaacgttcttcccccattggcacaaggattgttatactgagtctacagaaaaaagaagtaatcctcagtacaccatatggctctacagtgaacgatatttaaagtcataataatgtaaattctcttgattctcaagtcttagaaccaaccaatagacaaagcacagaagacttgatgatggttggagaacagactataaattgctatccactgaataatataagagctatagctggaaatggctgggaggtgaaagcagaagaaaagagaggtgggcattgctatctccagcttgcatggacaagagggcagttaaataacactgtgcacagtggatggaacaagaacacaaggtttagggatttatcaaaatcacaaaggtaatagaagaactaaaaataatttactataaaaatttggaagtgatagaattaggtaaattctctttcttcaatagggaaatcaatgtatgacatctaacactgataaagcaaaaaacagtgggtaaaagtggtcatgggttgacagttgttgcagctggggatgtgtacagggggttggttatactattctccttttatacataattgaaagttttcataataaaaagagaagaaaaaataatgaatcaagcaatctttggaattataatgatagtcactggaaaaactaaaaataaaaatggaagatggtttgctgggaatgggacttggactaaggaggggtgaaacagggctggagactttcggctttccattataagtctgtgtgtgtgtgtgtgtgtgtgtgtgtgtgtgtgtgtgtgtgtgtgtgtgtgtgtgtgtgtgtgtgtgtgtgttatgtgcataaatgaagttttttcattaaagaaataacatttaaaagacaataactgttggtgaagatgtagaaaaattggaaccctcctacattgctggagtgattgaaaaatggtgtagtcactttggaaaacaattggcagttcctcaaaaggataaaatagtttacgatccagcaattccaatcctaggtatatacccaagggaactaggaacacatgtccacacaaaaacctctacacaaatgttcatagcaacattattcataatagcaaaaaagtagaagtggctgggaacagtggttcacacctataatcccagcactttgggaggccgaggcaggaggatcacttgaggccaggagttcaagaccagcctgggcaacaaggcaaaaccctgtctctacaaaaaactagctgagcatggtggcacatgcctgtagtcccagctactggagaggctgaggtgggaggatggctcaagcctgggaggttgaggctacaatgagctatgactgcgctgctgtactccagcctgagcgacagaataagaccctgtctcaaagaa

SEQ ID NO: 4 - DNA, 101 bp, Homo sapiens
  misc_feature (51)..(51): s is G or C
cctgtcctgaatggagtttttccccctgtacaactgtcatcaacctgttcsggccctctcactgacaggcaggtccctacctatatttgaggggcagccca

SEQ ID NO: 5 - DNA, 625 bp, Homo sapiens
  misc_feature (492)..(492): n is G or C
gacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctntcaccagatgagattaacaccatctatcttggcgggcccttcagtcctaatgtcctgaactggcgggcactgaagtatgaagtgcaaggcgaagtgttcaccaaaccccagctgtggccctgaggcccagctg

SEQ ID NO: 6 - protein, 204 aa, Homo sapiens
DMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 7 - protein, 153 aa, Homo sapiens [sequence omitted]

SEQ ID NO: 8 - DNA, 101 bp, Homo sapiens
  misc_feature (51)..(51): m is A or C
ctgttcgggccctctcactgacaggcaggtccctacctatatttgaggggmagcccattgcatttctggacagctctcgccattagggtgcacacacgcac

SEQ ID NO: 9 - DNA, 101 bp, Homo sapiens
  misc_feature (51)..(51): y is T/U or C
gtgactctctgggaggtgcccagggcagagggtgactccaggatgggatgyctttgcagtgggtgatggtcttcaagaaccagtctcaaacttgtagtgac

SEQ ID NO: 10 - DNA, 101 bp, Homo sapiens
  misc_feature (51)..(51): r is G or A
cgcaaaggacgagagagtcttggaagccaggggaggtgggtgggagtcagrgtctcggcattcttctccatccagagcctagggtttaaggctggaaggat

SEQ ID NO: 11 - DNA, 101 bp, Homo sapiens
  misc_feature (51)..(51): r is G or A
agtcagggtctcggcattcttctccatccagagcctagggtttaaggctgraaggatatttacggagggtaatgactttgtaaacaagacctagaaatcta

SEQ ID NO: 12 - DNA, 101 bp, Homo sapiens
  misc_feature (51)..(51): r is G or A
aggctggaaggatatttacggagggtaatgactttgtaaacaagacctagraatctatctcccagtagcatcatctttgacccatgtcctctctgcccagc

SEQ ID NO: 13 - DNA, 101 bp, Homo sapiens
  misc_feature (51)..(51): y is T/U or C
gagccacacattcaggacagcctgcgaggcaggtgcacagtgcggatggcygtcccgggccccactaggggtctgcagctcgccacttcctcagccaccgc

SEQ ID NO: 14 - DNA, 101 bp, Homo sapiens
  misc_feature (51)..(51): m is A or C
ccacacattcaggacagcctgcgaggcaggtgcacagtgcggatggccgtmccgggccccactaggggtctgcagctcgccacttcctcagccaccgccga

SEQ ID NO: 15 - DNA, 101 bp, Homo sapiens
  misc_feature (51)..(51): y is T/U or C
gtgtgccctgggctggagagggcgtggggctctccagggatgagtgacacyttactgagccacagaaccgagctgcagctggtggcgacagggaaggggcc

SEQ ID NO: 16 - DNA, 101 bp, Homo sapiens
  misc_feature (51)..(51): r is G or A
gggcgtggggctctccagggatgagtgacaccttactgagccacagaaccragctgcagctggtggcgacagggaaggggccctgtcaggaatgagaaagg

SEQ ID NO: 17 - DNA, 101 bp, Homo sapiens
  misc_feature (51)..(51): s is G or C
acaccttactgagccacagaaccgagctgcagctggtggcgacagggaagsggccctgtcaggaatgagaaaggaagacagaacaaggtaggtaagtgtca

SEQ ID NO: 18 - DNA, 101 bp, Homo sapiens
  misc_feature (51)..(51): w is A or T/U
gggaggaagatgctggccatggggcccagggcggggagccccttggcaccwcgggaaccccagcccaggaggtttcactggaagagaggctgggcttgagt

SEQ ID NO: 19 - DNA, 101 bp, Homo sapiens
  misc_feature (51)..(51): y is T/U or C
gctggccccctgtcctgcgcaggacgagaaggagcaggccccaaggggacyaggcccgcatcttatttccttgtgaccgaggcagcagcttgaatgaagcc

SEQ ID NO: 20 - DNA, 104 bp, Homo sapiens
ggggaccaggcccgcatcttatttccttgtgaccgaggcagcagcttgaatgaagcctggccttcacctggcccagcggcttatcgctttttcattagggtcaa

SEQ ID NO: 21 - DNA, 101 bp, Homo sapiens
  misc_feature (51)..(51): y is T/U or C
cccagcggcttatcgctttttcattagggtcaaagagaaatgtcttctgcytatgcccttcatctagctggtgcattgtaacacactccaaacctgtttcc

SEQ ID NO: 22 - DNA, 101 bp, Homo sapiens
  misc_feature (51)..(51): m is A or C
cccacagcccgatggcctgttggccacacagtccacaactccctgcccagmccagggccctgtgccagaggagctgtggtgatgccacattggaaaaggca

SEQ ID NO: 23 - DNA, 101 bp, Homo sapiens
  misc_feature (51)..(51): y is T/U or C
cagagcatatagccgggggctcagggtgaaactagtcccagcttcaagccycagctgtgtccctgacttctagtgttgttacttcagccacgtctcagtgg

SEQ ID NO: 24 - DNA, 101 bp, Homo sapiens
  misc_feature (51)..(51): r is G or A
cttcaagccccagctgtgtccctgacttctagtgttgttacttcagccacrtctcagtggctccgtggaaatggagctccactcattcattcaacaagtat

SEQ ID NO: 25 - DNA, 101 bp, Homo sapiens
  misc_feature (51)..(51): r is G or A
gccacgtctcagtggctccgtggaaatggagctccactcattcattcaacragtatggattgaggccttgcctgggtgccagaccctgctctaggtgctgg

SEQ ID NO: 26 - DNA, 101 bp, Homo sapiens
  misc_feature (51)..(51): y is T/U or C
actcagtgtgtcgggcggtggtagccgctaccgtgggaaatagaacagtcyggggctgcacagcagggagagtgctgtgttatgcgaggaggttggagaaa

SEQ ID NO: 27 - DNA, 101 bp, Homo sapiens
  misc_feature (51)..(51): r is G or A
tgctgtgttatgcgaggaggttggagaaatcctccccatgagataagatgrgaacagagatcgggacgaaacaaggggaggggacacccatgcacagatcg

SEQ ID NO: 28 - DNA, 101 bp, Homo sapiens
  misc_feature (51)..(51): m is A or C
catgagataagatgggaacagagatcgggacgaaacaaggggaggggacamccatgcacagatcggggcaaagtcatcaagggaaagggaactgcaggtgc

SEQ ID NO: 29 - DNA, 101 bp, Homo sapiens
  misc_feature (51)..(51): r is G or A
aacaaggggaggggacacccatgcacagatcggggcaaagtcatcaagggraagggaactgcaggtgctaaggtcttgagcaagagcgagctggggatgcc

SEQ ID NO: 30 - DNA, 101 bp, Homo sapiens
  misc_feature (51)..(51): w is A or T/U
agatcggggcaaagtcatcaagggaaagggaactgcaggtgctaaggtctwgagcaagagcgagctggggatgcccttcccagcactcacgggtggccctc

SEQ ID NO: 31 - DNA, 101 bp, Homo sapiens
  misc_feature (51)..(51): s is G or C
gatgcccttcccagcactcacgggtggccctcgggctggagcagaggccasggcaagggcagcaggagcccagcagggcctcagaggccaggaccgggctt

SEQ ID NO: 32 - DNA, 101 bp, Homo sapiens
  misc_feature (51)..(51): r is G or A
ctaagtggaatggagtgatgtggtctgactcacattttaacaaagtcaccrtggctgctgtgtggagaacgcaacgcagggcgaaggcggagcagggagat

SEQ ID NO: 33 - DNA, 101 bp, Homo sapiens
  misc_feature (51)..(51): r is G or A
aggcggagcagggagatgagcgccaaggtgcctgcagagatgctggtgggragcgatggtgcttgtggtagtggaggactggagaggggcgtgtgtgtaag

SEQ ID NO: 34 - DNA, 103 bp, Homo sapiens
agatgcatgtatacattatactgctatcgtatacattatttacttaaatagtatattatataatccataaagtatagatataaatatataatatataaatgtg

SEQ ID NO: 35 - DNA, 101 bp, Homo sapiens
  misc_feature (51)..(51): k is G or T/U
atccataaagtatagatataaatatataatatataaatgtgcatatatgtkaatatttacatttgctatgcaattacaattttaaaatcttctattacaga

SEQ ID NO: 36 - DNA, 101 bp, Homo sapiens
  misc_feature (51)..(51): w is A or T/U
taaatgtgcatatatgttaatatttacatttgctatgcaattacaattttwaaatcttctattacagaaaatttcaaatatacacgaaagtcactcccaag

SEQ ID NO: 37 - DNA, 101 bp, Homo sapiens
  misc_feature (51)..(51): r is G or A
cactcccaagcatccattgcccagcgtcaacaatgagcaactcatggccarttttgtcaattttgttccatctctacccccgccccttccccaccaccacc

SEQ ID NO: 38 - DNA, 101 bp, Homo sapiens
  misc_feature (51)..(51): r is G or A
aatccgagatgtcacatcagtgaaaggagaggctttggggtggcttggatrtgagcgtgtgaggtcttcccgtgttgtgtgtgaacaactgcaaggatgaa

SEQ ID NO: 39 - DNA, 101 bp, Homo sapiens
  misc_feature (51)..(51): m is A or C
aggacaaactcctacaacagcagaatgtctgggtgtcctgatgtctcatamcaatatatgctttcaagctatttacagtaatgctttgatgtacttactca

SEQ ID NO: 40 - DNA, 101 bp, Homo sapiens
  misc_feature (51)..(51): y is T/U or C
ctttgatgtacttactcactaaaaggccaaggataaaacattatttaaatyaacaaagtactaaattttgtgacactgtcagcccacctgcacatagacat

SEQ ID NO: 41 - DNA, 101 bp, Homo sapiens
  misc_feature (51)..(51): w is A or T/U
ttttgtgacactgtcagcccacctgcacatagacataatgtagcttggctwttgtatagacaagacccctatataagaaaagctttaagcagacaaggcgt

SEQ ID NO: 42 - DNA, 101 bp, Homo sapiens
  misc_feature (51)..(51): k is G or T/U
cacgactcaccttgaattccttcctgtgcgagatccaagaactctctcggkgtctgaatcgggacccctttttccagcaacactgtgttcttccccttggc

SEQ ID NO: 43 - DNA, 101 bp, Homo sapiens
  misc_feature (51)..(51): r is G or A
cttgaattccttcctgtgcgagatccaagaactctctcggggtctgaatcrggacccctttttccagcaacactgtgttcttccccttggcttagtcaccc

SEQ ID NO: 44 - DNA, 101 bp, Homo sapiens
  misc_feature (51)..(51): y is T/U or C
cccctcagctggtggctcccacccgccgcctgccctctcccctgcctcctycccaagccagagacctggggccacctgcgacttcctgctctccctcaccc

SEQ ID NO: 45 - DNA, 101 bp, Homo sapiens
  misc_feature (51)..(51): r is G or A
acactgtgctgggccaaatgactcaagagcccacagctctccctgccaccrtcccacccgattcgcgccagctgcctctgtcacgggcgcctgcaatgctc

SEQ ID NO: 46 - DNA, 103 bp, Homo sapiens
gagactgggctgcattgtgcgacctgtattcctttctcctagttgactgccgcgtttctgactcctttgaagcgagcatctggcttttccaattagatgaagg

SEQ ID NO: 47 - DNA, 101 bp, Homo sapiens
  misc_feature (51)..(51): k is G or T/U
cgtttctgactcctttgaagcgagcatctggcttttccaattagatgaagkctgacaagctgtggaggggagggcggcagataccatgtacctggtcattc

SEQ ID NO: 48 - DNA, 101 bp, Homo sapiens
  misc_feature (51)..(51): y is T/U or C
cattcagactaggggtgtccttgagcagactcatggtgtggaagtcagacygggagtctcctggagcagactcacagtgtagggggtcagcagaggcagca

SEQ ID NO: 49 - DNA, 101 bp, Homo sapiens
  misc_feature (51)..(51): m is A or C
gcctgcccctccagccctcctgacattcttagaagcacctactttcctgcmtgaaatcctttcctgactaaagcacccacagctgtgtctgttccctgtaa

SEQ ID NO: 50 - DNA, 101 bp, Homo sapiens
  misc_feature (51)..(51): r is G or A
aactcaaagacggaggaggcacatccaggaggaacctgtggggagggcccrtctggccagatctccactgccctgtccagacttgggcttgcctaatagat

SEQ ID NO: 51 - DNA, 101 bp, Homo sapiens
  misc_feature (51)..(51): w is A or T/U
catgagacagggacagctatgacccctatttcacagatgaggctgagtagwgtgcccaaggtcacacagccaggaggcacagcagccaggcctgacagacc

SEQ ID NO: 52 - DNA, 101 bp, Homo sapiens
  misc_feature (51)..(51): r is G or A
tgacacatgccaagtactttccatggaaccactcacttgctcctcacaagraagagccacattattcccatttcacaggtgagaaaatcgagacccagaga

SEQ ID NO: 53 - DNA, 101 bp, Homo sapiens
  misc_feature (51)..(51): r is G or A
catggtcacagagttgataagggctcatttgctggactcccaaacgcagtrctcataactgctacgttccagggcctgaaggaaaaactctgcatccatgg

SEQ ID NO: 54 - DNA, 101 bp, Homo sapiens
  misc_feature (51)..(51): y is T/U or C
cccaggaggtagccccaaattcctagggatttcccaagtgataggagtatyttattattcatggtggtctctgagagtttatgcgagtgaagtgactcatg

SEQ ID NO: 55 - DNA, 101 bp, Homo sapiens
  misc_feature (51)..(51): r is G or A
gggccctaggtagtttttgctgacaatacgaccatggaggggctggccacrccactgaggttctgtgatatcagcctggcctcccggaaggagaccaggaa

SEQ ID NO: 56 - DNA, 101 bp, Homo sapiens
  misc_feature (51)..(51): s is G or C
acggggaagacaatgagagctttgtgtttgggcccctcctccatctcgccsctgtgtctcctcttttggctggttctgatttgaatgcttttgttatgata

SEQ ID NO: 57 - DNA, 101 bp, Homo sapiens
  misc_feature (51)..(51): y is T/U or C
atttagtttggtaaccttgctgcccagtcccaatggaaggatcctaaatayggtctaaggacctcctgatacaattatccagattctctccttcacagaac

SEQ ID NO: 58 - DNA, 101 bp, Homo sapiens
  misc_feature (51)..(51): y is T/U or C
tccttcacagaacttgaggcactgcgataagatccaaaactatatatacayagtggaatactatacagccttaaaaaagaaggaaacgctgtcattcacca

SEQ ID NO: 59 - DNA, 101 bp, Homo sapiens
  misc_feature (51)..(51): r is G or A
aactatatatacacagtggaatactatacagccttaaaaaagaaggaaacrctgtcattcaccacaacatggatgaacctggaggacattatgctaagtga

SEQ ID NO: 60 - DNA, 101 bp, Homo sapiens
  misc_feature (51)..(51): r is G or A
atactatacagccttaaaaaagaaggaaacgctgtcattcaccacaacatrgatgaacctggaggacattatgctaagtgaaaaaatccaggcacagacag

SEQ ID NO: 61 - DNA, 101 bp, Homo sapiens
  misc_feature (51)..(51): y is T/U or C
tccaggcacagacagacaaataccacatgatctcacgcatatgtgaaatayaaaaaagccaaactcagggaggcagagagtaggatgatggtcaccagggg

SEQ ID NO: 62 - DNA, 101 bp, Homo sapiens
  misc_feature (51)..(51): y is T/U or C
accctgtctctattaaaaatacaaaaattagctgggcgtggtggcaggcayctgtaatcccagctactcgggaggctgaggcaggagaattgcttgagctc

SEQ ID NO: 63 - DNA, 101 bp, Homo sapiens
  misc_feature (51)..(51): y is T/U or C
aggagaattgcttgagctcaggaggcagaggttgcagtgagctgagactgygccattgcactccagtctgggcaacagagcaagactctatctcaaaataa

SEQ ID NO: 64 - DNA, 101 bp, Homo sapiens
  misc_feature (51)..(51): r is G or A
tggttcacgcctataatcccagcactttgggaggctgaggtgggtggatcrttttgaggccaggagttcgacaccagcctagccaacatggtgaaacccca

SEQ ID NO: 65 - DNA, 101 bp, Homo sapiens
  misc_feature (51)..(51): y is T/U or C
gggtggtgaggggggggcggggaagtggaacagaggcaaaaccttagcaayacacatttttagatgatcttccagaataaattcatagggaggcccaggca

SEQ ID NO: 66 - DNA, 101 bp, Homo sapiens
  misc_feature (51)..(51): y is T/U or C
gaggcccaggcacagtggctcacgcctgtaatcccagcactttgggaggcygaggcaggcggatcacgaggtcaggagatggagaccatcctggctaacac

SEQ ID NO: 67 - DNA, 101 bp, Homo sapiens
aaaaattagccgggcgtggtggcaggtgcctgtagtcccagctactcggggaggctgaggcaggagaacggcatgaacccaggaggcggagcttgcagtga

SEQ ID NO: 68 - DNA, 101 bp, Homo sapiens
  misc_feature (51)..(51): y is T/U or C
gtggcaggtgcctgtagtcccagctactcggggaggctgaggcaggagaayggcatgaacccaggaggcggagcttgcagtgaactaagatcacgccactg

SEQ ID NO: 69 - DNA, 101 bp, Homo sapiens
  misc_feature (51)..(51): r is G or A
caggtgcctgtagtcccagctactcggggaggctgaggcaggagaacggcrtgaacccaggaggcggagcttgcagtgaactaagatcacgccactgcact

SEQ ID NO: 70 - DNA, 101 bp, Homo sapiens
  misc_feature (51)..(51): k is G or T/U
tgtagtcccagctactcggggaggctgaggcaggagaacggcatgaaccckggaggcggagcttgcagtgaactaagatcacgccactgcactccagcctg

SEQ ID NO: 71 - DNA, 101 bp, Homo sapiens
  misc_feature (51)..(51): r is G or A
ccagctactcggggaggctgaggcaggagaacggcatgaacccaggaggcrgagcttgcagtgaactaagatcacgccactgcactccagcctgggtgaca

SEQ ID NO: 72 - DNA, 101 bp, Homo sapiens
  misc_feature (51)..(51): y is T/U or C
aggagaacggcatgaacccaggaggcggagcttgcagtgaactaagatcaygccactgcactccagcctgggtgacagagcaagattccatctcaaaaaaa

SEQ ID NO: 73 - DNA, 101 bp, Homo sapiens
  misc_feature (51)..(51): y is T/U or C
aagaaattcatagggaaaagaaggtcagagaccaagggaagggaaggttcygggagaaaagcagggggcaggcagggcccaagaatcctgctgcccatgag

SEQ ID NO: 74 - DNA, 101 bp, Homo sapiens
  misc_feature (51)..(51): r is G or A
ttattgtgtccacatgaattcaataaaaaacaagcagggcgggtggtgggrcactgactaggagggctgatttgtaagttggtaagactgtagctcttttt

SEQ ID NO: 75 - DNA, 101 bp, Homo sapiens
  misc_feature (51)..(51): k is G or T/U
gcgggtggtggggcactgactaggagggctgatttgtaagttggtaagackgtagctctttttcctaattagctgaggatgtgtttaggttccattcaaaa

SEQ ID NO: 76 - DNA, 101 bp, Homo sapiens
  misc_feature (51)..(51): y is T/U or C
agctctttttcctaattagctgaggatgtgtttaggttccattcaaaaagygggcattcctggccaggcatggtggctcacacctgtaatctcagagcttt

SEQ ID NO: 77 - DNA, 101 bp, Homo sapiens
  misc_feature (51)..(51): y is T/U or C
cctgatacgacctgtccttctcaaaacacctaaacttgggagaacattgtyccccagtgctggggtaggagagtctgcctgttattctgcctctatgcaga

SEQ ID NO: 78 - DNA, 101 bp, Homo sapiens
  misc_feature (51)..(51): y is T/U or C
agatatctttgtcagcattgcatcgttagcttctcctgataaactaattgyctcacattgtcactgcaaatcgacacctattaatgggtctcacctcccaa

SEQ ID NO: 79 - DNA, 101 bp, Homo sapiens
  misc_feature (51)..(51): r is G or A
ctattaatgggtctcacctcccaactgcttccccctctgttcttcctgctrgcatgtgccggcaactttgtccacggacacaagtgcgatatcaccttaca

SEQ ID NO: 80 - DNA, 101 bp, Homo sapiens
  misc_feature (51)..(51): y is T/U or C
cccatacatttctgaaaaaccaaatactctcacagaaacagatattgagaygacaggttgagggagctttcattttgtctaagagacttcctatggcaaca

SEQ ID NO: 81 - DNA, 101 bp, Homo sapiens
  misc_feature (51)..(51): y is T/U or C
tggccacctaacagccctctgggttccggggctggccgtccagagctcctyagcttgctctggccggccgaactcccctccagctcggtctggaaccatcc

SEQ ID NO: 82 - DNA, 101 bp, Homo sapiens
  misc_feature (51)..(51): y is T/U or C
ccgtccagagctcctcagcttgctctggccggccgaactcccctccagctyggtctggaaccatcctgctgggcagcgtccagcacatccctgcttcgggc

SEQ ID NO: 83 - DNA, 101 bp, Homo sapiens
  misc_feature (51)..(51): y is T/U or C
ctccaagtccatccaaccgagatggctcctactagaagccaagagtccacygggttgagcactgggtctctggaggcctgtggcactgctgagaaggctct

SEQ ID NO: 84 - DNA, 101 bp, Homo sapiens
  misc_feature (51)..(51): r is G or A
ctagaagccaagagtccaccgggttgagcactgggtctctggaggcctgtrgcactgctgagaaggctctaacaaagccaagggaagggccacctcactag

SEQ ID NO: 85 - DNA, 101 bp, Homo sapiens
accgggttgagcactgggtctctggaggcctgtggcactgctgagaaggctctaacaaagccaagggaagggccacctcactagaagccaggcctggagga

SEQ ID NO: 86 - DNA, 101 bp, Homo sapiens
  misc_feature (51)..(51): y is T/U or C
tttagacccagctctgccactgactagctgtgtggctggccttcagcacaycttcacacctctctgcacctcagtttccacatgtgaagatatgaaagtga

SEQ ID NO: 87 - DNA, 101 bp, Homo sapiens
  misc_feature (51)..(51): y is T/U or C
attctaagaaggaggggaccgggttggaaaggatcaaaccatccaggatgycgagtctggggcaatccatctgggctgtttctggaagacccccgggtgca

SEQ ID NO: 88 - DNA, 101 bp, Homo sapiens
  misc_feature (51)..(51): y is T/U or C
ttctaagaaggaggggaccgggttggaaaggatcaaaccatccaggatgcygagtctggggcaatccatctgggctgtttctggaagacccccgggtgcag

SEQ ID NO: 89 - DNA, 101 bp, Homo sapiens
  misc_feature (51)..(51): k is G or T/U
caagctggctggctgatgaagggtttcttgggtggacaagtagttggagckatttcctatttaccaaagagagctaaagttcataattctacagagagttc

SEQ ID NO: 90 - DNA, 101 bp, Homo sapiens
  misc_feature (51)..(51): w is A or T/U
tcttgggtggacaagtagttggagctatttcctatttaccaaagagagctwaagttcataattctacagagagttccataatgaacctcaaatacctctgt

SEQ ID NO: 91 - DNA, 101 bp, Homo sapiens
  misc_feature (51)..(51): k is G or T/U
cataattctacagagagttccataatgaacctcaaatacctctgttttttkaaggagtttctcatatacagcactagctgactatcctgggcaggatggga

SEQ ID NO: 92 - DNA, 101 bp, Homo sapiens
ggagtttctcatatacagcactagctgactatcctgggcaggatgggagataatgaatgcagtgccaatcgggctggatttatatggtcctcagtgaggct

SEQ ID NO: 93 - DNA, 101 bp, Homo sapiens
  misc_feature (51)..(51): s is G or C
gggccacggcagggatggagtgtcgccccctcgctgcgataccagagcaastaaaacgttaaggccttgcactaaagctgcccttaggatgcattctttta

SEQ ID NO: 94 - DNA, 101 bp, Homo sapiens
  misc_feature (51)..(51): s is G or C
agactcttttcaattcttattttatccttgtttcctttagaaagtcctttsaaaaatatctttagagggttttttcctatactatgtggccatatacgggt

SEQ ID NO: 95 - DNA, 101 bp, Homo sapiens
  misc_feature (51)..(51): r is G or A
ccaaagatactgacacactcttacatttccaactagaatcaggaacgaggrgtgactctcagtcagttcattaagtaaatgtctttctaaccgctctgccc

SEQ ID NO: 96 - DNA, 101 bp, Homo sapiens
  misc_feature (51)..(51): m is A or C
tcagttcattaagtaaatgtctttctaaccgctctgcccatgggacatcamgccccacaggggaaaggggaagcttctgtagcctgggattctggtgcctc

SEQ ID NO: 97 - DNA, 101 bp, Homo sapiens
  misc_feature (51)..(51): r is G or A
agcctgggattctggtgcctcagtctgggtctagactttcctgaaaaaacrttaaaatatgaactgcattcctagaatttagcctacataaataagagatg

SEQ ID NO: 98 - DNA, 101 bp, Homo sapiens
  misc_feature (51)..(51): s is G or C
gatgaacacaaagatttctatagtttactcactgccgcttatttacagaascaaaaatctgccacgataggggcctgacaaatgacagtaccactgtgcaa

SEQ ID NO: 99 - DNA, 101 bp, Homo sapiens
  misc_feature (51)..(51): m is A or C
acaaatgacagtaccactgtgcaatgcgtttctacgcagctctcaatcccmtgttctctaataccaccgaagggcttaggaaatgcttatggtatatgtaa

SEQ ID NO: 100 - DNA, 102 bp, Homo sapiens
atgttctctaataccaccgaagggcttaggaaatgcttatggtatatgtaaagagtaaagaagttacaaaacagtatcaacagttgacccctattttaaaaa

SEQ ID NO: 101 - DNA, 101 bp, Homo sapiens
  misc_feature (51)..(51): r is G or A
taaaaagtatttttaaaagtgtgacgatatttaccaaaatattaacagcartagttacctctggctggtgggatgagtgaatgtatttttgttgaatatat

SEQ ID NO: 102 - DNA, 101 bp, Homo sapiens
  misc_feature (51)..(51): y is T/U or C
aaaaatatgtaagaacttgaaagctgcttggacagcgctgctgatagaaayccctgagcatcttgtcactgttcttctgattcagagggtctgggtggggc

SEQ ID NO: 103 - DNA, 101 bp, Homo sapiens
  misc_feature (51)..(51): r is G or A
gaaacccctgagcatcttgtcactgttcttctgattcagagggtctgggtrgggcaggggtggtctgagattctgtatttctaagaagctcccagtgatgt

SEQ ID NO: 104 - DNA, 101 bp, Homo sapiens
  misc_feature (51)..(51): k is G or T/U
gttcttctgattcagagggtctgggtggggcaggggtggtctgagattctktatttctaagaagctcccagtgatgtccatgctgctggtccatggaccac

SEQ ID NO: 105 - DNA, 101 bp, Homo sapiens
  misc_feature (51)..(51): y is T/U or C
tgcccaaggctcagatcaatggcacctgctcccagagcctcctctgatctyccaggacacctttccctgatctgtgcacttatctcttgctgcctggcaaa

SEQ ID NO: 106 - DNA, 101 bp, Homo sapiens
  misc_feature (51)..(51): r is G or A
tgtgcacttatctcttgctgcctggcaaaatgtcttagctcctcacttggrccatgtgctgctctcctctcccatggggagagccacacggagagtgctgg

SEQ ID NO: 107 - DNA, 101 bp, Homo sapiens
  misc_feature (51)..(51): y is T/U or C
ggagagtgctggccaaagcagcagagttcaggccaaaggatgtgcactcayttattcaacaggcatgcaggatttccagggaaagctggattttaaaacct

SEQ ID NO: 108 - DNA, 101 bp, Homo sapiens
  misc_feature (51)..(51): y is T/U or C
tttattcaacaggcatgcaggatttccagggaaagctggattttaaaaccyctgggaacaagagcagaacctgactgagagctcatgtgggcacttttcat

SEQ ID NO: 109 - DNA, 101 bp, Homo sapiens
  misc_feature (51)..(51): y is T/U or C
gctcatgtgggcacttttcatagcagaatagctcatgaggtatagagacayggacgcagaacgtgggctgtagcgacagatggtcctgcattctagtcccc

SEQ ID NO: 110 - DNA, 123 bp, Homo sapiens
gctgaagttccacaagtgctgggtgtggttctaggtgctgaggacgtgtcactaaagacacgcaggccgagtccctgttctcatggaatgttctaatgggagagttagaaaaacaaacatgta

SEQ ID NO: 111 - DNA, 101 bp, Homo sapiens
  misc_feature (51)..(51): r is G or A
agctttgggcaggacaaagactgtacaatctgatttacgtgatttaaaagrgtcagtctggctactgtgtggtaaataggctgaaagggggaaagcataga

SEQ ID NO: 112 - DNA, 101 bp, Homo sapiens
  misc_feature (51)..(51): r is G or A
ctgaaagggggaaagcatagaagcaagatggcctgttgggaggctaccacrgtaaaccaggctagagatgatggtggcgtggacagaatgaagcaagatgg

SEQ ID NO: 113 - DNA, 101 bp, Homo sapiens
  misc_feature (51)..(51): y is T/U or C
tgaagcaagatggcctgttgggaggctaccacagtaaaccaggctagagaygatggtggcgtggacagaatgaagcaagatggcctgttgggaggctacca

SEQ ID NO: 114 - DNA, 101 bp, Homo sapiens
  misc_feature (51)..(51): m is A or C
cagatttgggatatgactaaaaataaaaccagaaggatttgctgacagatmggttgtagggggtaagatacaggggaggaaaagatgacctctttgttcct

SEQ ID NO: 115 - DNA, 101 bp, Homo sapiens
  misc_feature (51)..(51): s is G or C
tgctgacagatcggttgtagggggtaagatacaggggaggaaaagatgacstctttgttcctgcccaaacccctctggcgatggtcagtactgtttacaga

SEQ ID NO: 116 - DNA, 101 bp, Homo sapiens
  misc_feature (51)..(51): r is G or A
gaccagtgaggcaggagaacactggagagtgggcggtaccccaggaagccrgtgaggacactcaaggagggagggttgactgtgtcaaatgtactgaaagg

SEQ ID NO: 117 - DNA, 101 bp, Homo sapiens
  misc_feature (51)..(51): k is G or T/U
accagtgaggcaggagaacactggagagtgggcggtaccccaggaagccgktgaggacactcaaggagggagggttgactgtgtcaaatgtactgaaagga

SEQ ID NO: 118 - DNA, 101 bp, Homo sapiens
  misc_feature (51)..(51): y is T/U or C
gcctgggggtagcatctggaatccttcccacctttagggctgctgtgctgyggtgctgctgtttaatggcacaggagggccacatgactgaatctctctca

SEQ ID NO: 119 - DNA, 101 bp, Homo sapiens
  misc_feature (51)..(51): s is G or C
ctgctgtgctgcggtgctgctgtttaatggcacaggagggccacatgactsaatctctctcagcagtccaggcagtcatgcagaaggcccagtagagcacc

SEQ ID NO: 120 - DNA, 100 bp, Homo sapiens
  misc_feature (51)..(51): s is G or C
tgagcaagcacctagctgtgacacacctctccagagactgcactccccccsgcgccacccaccccaaaagcagataggtaatggtatacagtaaccattt

SEQ ID NO: 121 - DNA, 101 bp, Homo sapiens
  misc_feature (51)..(51): r is G or A
ctgtgacacacctctccagagactgcactcccccccgcgccacccaccccraaagcagataggtaatggtatacagtaaccatttctagaagtgtaagtag

SEQ ID NO: 122 - DNA, 101 bp, Homo sapiens
  misc_feature (51)..(51): r is G or A
gttcaggtgacaagtgccacagtaggcttgatcaagtagacaggcaggcargacaaatgcttaccaatgcaagctaatgaaatgtttcttttgcagaattc

SEQ ID NO: 123 - DNA, 101 bp, Homo sapiens
  misc_feature (51)..(51): m is A or C
agacaggcaggcaagacaaatgcttaccaatgcaagctaatgaaatgtttmttttgcagaattcctgtcctgtgaaggaagccaaccagagtacgttggaa

SEQ ID NO: 124 - DNA, 101 bp, Homo sapiens
  misc_feature (51)..(51): s is G or C
cattgctatctccagcttgcatggacaagagggcagttaaataacactgtscacagtggatggaacaagaacacaaggtttagggatttatcaaaatcaca

SEQ ID NO: 125 - DNA, 101 bp, Homo sapiens
  misc_feature (51)..(51): y is T/U or C
tcatgggttgacagttgttgcagctggggatgtgtacagggggttggttayactattctccttttatacataattgaaagttttcataataaaaagagaag

SEQ ID NO: 126 - DNA, 101 bp, Homo sapiens
  misc_feature (51)..(51): r is G or A
tgttcatagcaacattattcataatagcaaaaaagtagaagtggctgggarcagtggttcacacctataatcccagcactttgggaggccgaggcaggagg

SEQ ID NO: 127 - DNA, 101 bp, Homo sapiens
  misc_feature (51)..(51): r is G or A
attcataatagcaaaaaagtagaagtggctgggaacagtggttcacacctrtaatcccagcactttgggaggccgaggcaggaggatcacttgaggccagg

SEQ ID NO: 128 - DNA, 101 bp, Homo sapiens
  misc_feature (51)..(51): m is A or C
aggcaggaggatcacttgaggccaggagttcaagaccagcctgggcaacamggcaaaaccctgtctctacaaaaaactagctgagcatggtggcacatgcc

(19) United States
(12) Patent Application Publication
(10) Pub. No.: US 2007/0065863 A1
(43) Pub. Date: Mar. 22, 2007

(54) Method for detecting a risk of cardiovascular disease

(76) Inventors: Ulrich Broeckel, Wauwatosa, WI (US); Karen Maresso, Midlothian, IL (US)

Correspondence Address: QUARLES & BRADY LLP, 411 E. WISCONSIN AVENUE, SUITE 2040, MILWAUKEE, WI, 53202-4497, US

(21) Appl. No.: 11/524,568

(22) Filed: Sep. 21, 2006

Related U.S. Application Data
(60) Provisional application No. 60/719,727, filed on Sep. 22, 2005.

(51) Int. Cl. C12Q 1/68

(52) U.S. Cl. 435/6

(57) ABSTRACT

Single nucleotide polymorphisms associated with serum C-reactive protein levels are disclosed. Also disclosed are methods of using these markers to predict the propensity for cardiovascular diseases and the time to first myocardial infarction.

### CROSS-REFERENCE TO RELATED APPLICATION

This application claims the benefit of U.S. patent application Ser. No. 60/719,727, filed on Sep. 22, 2005.

This invention was made with United States government support from the NHLBI Grant No. HL-R01 074321. The United States government has certain rights in this invention.

### BACKGROUND OF THE INVENTION

Serum C-reactive protein (CRP) level has emerged as a prognostic marker that is functionally linked to cardiovascular disease, in particular coronary artery disease (CAD) and myocardial infarction (MI) (Ridker PM, et al., N Engl J Med 2000;342(12):836-43; Buffon A, et al., J Am Coll Cardiol 1999;34(5):1512-21; Haverkate F, et al., Lancet 1997;349(9050):462-6; Ridker PM, et al., Circulation 1998;98(8):731-3; Ridker PM, et al., N Engl J Med 1997;336(14):973-9; and Koenig W, et al., Circulation 2004;109(11):1349-53). In general, a higher serum CRP level indicates a greater risk of cardiovascular disease. A significant portion of the inter-individual variability in serum CRP is determined by genetic factors (Pankow JS, et al., Atherosclerosis 2001;154(3):681-9.; Vickers MA, et al., Cardiovasc Res 2002;53(4):1029-34). Most recently, Ridker, et al. demonstrated that reduction of CRP levels to below 2 mg/L through the use of statins results in clinically significant improved event-free survival (Ridker P, et al., New Eng J Med 2005;352(1):20-8).

Using a genome wide scan we identified linkage of serum CRP levels to chromosomes 5 in a data-set of 513 Caucasian families with MI. In the present invention, we sought to identify polymorphisms which affect serum CRP levels and evaluate the impact with regard to clinical phenotypes.

### SUMMARY OF THE INVENTION

In one aspect, the present invention relates to a method of screening a human subject for propensity to develop a cardiovascular disease. The method involves (a) determining the status of a marker selected from single nucleotide polymorphism (SNP) marker IL4_{—}4135 or another SNP marker in linkage disequilibrium with IL4_{—}4135 in the genome of the human subject and (b) correlating the result from step (a) to the subject's propensity for developing a cardiovascular disease wherein on average subjects who carry the minor allele of IL4_{—}4135 are less likely to develop a cardiovascular disease than subjects who do not carry the minor allele of IL4_{—}4135.

In another aspect, the present invention relates to a method of correlating a human subject's serum or plasma C-reactive protein (CRP) level to the subject's genetic composition. The method involves (a) determining the status of a marker selected from single nucleotide polymorphism (SNP) marker IL4_{—}4135 or another SNP marker in linkage disequilibrium with IL4_{—}4135 in the genome of the human subject and (b) correlating the result from step (a) to the subject's serum or plasma CRP level wherein on average subjects who carry the minor allele of IL4_{—}4135 have a lower serum or plasma CRP level than subjects who do not carry the minor allele of IL4_{—}4135.

In another aspect, the present invention relates to a method of screening a human subject for propensity to develop a cardiovascular disease. The method involves (a) genotyping the genome of the human subject for SNP marker IL4_{—}4135 or another SNP marker that is in linkage disequilibrium with IL4_{—}4135, (b) genotyping the genome of the human subject for SNP marker CRP_{—}2667 or another SNP marker that is in linkage disequilibrium with CRP_{—}2667, and (c) correlating the result from steps (a) and (b) to the subject's propensity for developing a cardiovascular disease wherein individuals who are homozygous for the common allele of both IL4_{—}4135 and CRP_{—}2667 are more likely to develop a cardiovascular disease than individuals who are heterozygous for one and homozygous for the other of IL4_{—}4135 and CRP_{—}2667, who are in turn more likely to develop a cardiovascular disease than individuals who are homozygous for the minor allele of both IL4_{—}4135 and CRP_{—}2667.

In another aspect, the present invention relates to a method of correlating a human subject's serum or plasma CRP level to the subject's genetic composition. The method involves (a) genotyping the genome of the human subject for SNP marker IL4_{—}4135 or another SNP marker that is in linkage disequilibrium with IL4_{—}4135, (b) genotyping the genome of the human subject for SNP marker CRP_{—}2667 or another SNP marker that is in linkage disequilibrium with CRP_{—}2667, and (c) correlating the result from steps (a) and (b) to the subject's serum or plasma CRP level wherein on average individuals who are homozygous for the common allele of both IL4_{—}4135 and CRP_{—}2667 have a higher serum or plasma CRP level than individuals who are heterozygous for one and homozygous for the other of IL4_{—}4135 and CRP_{—}2667, who are in turn have a higher serum or plasma CRP level than individuals who are homozygous for the minor allele of both IL4_{—}4135 and CRP_{—}2667.

In still another aspect, the present invention relates to a method of screening a human subject for predicting time to first myocardial infarction (MI). The method involves (a) determining the status of a marker selected from SNP marker IL4_{—}1916 or another SNP marker in linkage disequilibrium with IL4_{—}1916 in the genome of the human subject and (b) correlating the result from step (a) to time to first myocardial infarction wherein subjects who carry the minor allele of IL4_{—}1916 are likely to have myocardial infarction for the first time at an older age than subjects who do not carry the minor allele of IL4_{—}1916.

### BRIEF DESCRIPTION OF THE DRAWINGS

FIGS. 1a-1n (SEQ ID NO:3) show the annotated DNA sequence for the IL4 region with SNPs. The amino acid sequence in FIGS. 1a-1n is provided in the sequence listing as SEQ ID NO:7.

FIGS. 2a-2k (SEQ ID NO:4) show SNPs in the IL4 region with flanking sequences, including a detailed description of the base pair changes using the IUPAC letters (i.e., R=A or G, Y=C or T, M=C or A, K=T or G, W=T or A, and S=G or C).

FIG. 3 shows that multipoint linkage analysis identified a region suggestive for linkage on chromosome 5 with a peak LOD score of 2.23 (p<0.001). Top panel: Ln CRP multipoint LOD plot for chromosome 5. Dashed line represent original linkage signal. Solid line represents LOD scores from conditional linkage analysis. Middle panel: Haploview plot for 45 SNPs in the candidate linkage region. Lower panel: P-values in full family set for selected SNPs with positive association.

FIG. 4 shows mean (±SD) InCRP levels by genotype (IL4_{—}4135 and CRP_{—}2667) in affected individuals.

FIG. 5A shows the difference in relative luciferase activity (RLU) for IL4_{—}4135 in both HepG2 and Jurkat T cells. RLU is adjusted for transfection efficiency. Significant higher RLU was detected for the minor/rare allele in both cell lines.

FIG. 5B shows the difference in relative luciferase activity (RLU) for IL4_{—}1916 in both HepG2 and Jurkat T cells. RLU is adjusted for transfection efficiency. Significant higher RLU was detected for the minor/rare allele in Jurkat T cells while there was no significant difference in HepG2 cells. This is consistent with published reports showing that the TFBS located at the IL4_{—}1916 plays a role in lymphocyte development.

FIG. 6 shows SNP_{—}2667 within the CRP gene.

### DETAILED DESCRIPTION OF THE INVENTION

Using a positional candidate gene approach, we evaluated single-nucleotide polymorphisms (SNPs) in candidate genes and intergenic regions within a region of human chromosome 5q31. The functional effects of these SNPs were evaluated using reporter-gene luciferase assays. Cox Proportional hazards modeling was used to demonstrate the ability of the SNPs to predict a clinical relevant phenotype, such as time to first myocardial infarction (MI).

In the present invention we disclose significant associations of SNPs within a haplotype block located in the IL4-IL13 intergenic region of human chromosome 5 with serum C-reactive protein (CRP) levels. These SNPs are IL4_{—}4135, IL4_{—}1916, IL4_{—}1158, IL4_{—}2243, and IL4_{—}3900. Given that serum CRP level is a prognostic marker that is functionally linked to cardiovascular diseases (Ridker PM, et al., N Engl J Med 2000;342(12):836-43; Buffon A, et al., J Am Coll Cardiol 1999;34(5):1512-21; Haverkate F, et al., Lancet 1997;349(9050):462-6; Ridker PM, et al., Circulation 1998;98(8):731-3; Ridker PM, et al., N Engl J Med 1997;336(14):973-9; and Koenig W, et al., Circulation 2004;109(11):1349-53), SNPs identified here are useful markers for assessing the risk of cardiovascular diseases such as stroke, coronary artery disease (e.g., myocardial infarction or heart attack), end stage renal disease, and peripheral artery disease. The identified SNPs can also serve as potential targets for drug development and diagnostic tools to investigate why patients respond differently to drugs affecting CRP levels.

In particular, we demonstrate that individuals who carry the minor allele of IL4_{—}4135 have on average a lower serum CRP level and thus a lower likelihood of developing a cardiovascular disease than individuals who do not carry the minor allele of IL4_{—}4135. By the minor allele (used interchangeably with the term “rare allele”) of IL4_{—}4135, we mean that the nucleotide at nucleotide position 4135 of the IL4-IL13 intergenic region of human chromosome 5 is “A” (Table 1). The corresponding position for the common allele of IL4_{—}4135 has a “G” (Table 1).

TABLE 1

| | | |
|---|---|---|
| GAGTGCTTGG TGAGTGGGAG GAAGATGCTG GCCATGGGGC CCAGGGCGGG | 1900 | var (1916):[A:0.03] |
| GAGCCCCTTG GCACCT/ACGGG AACCCCAGCC CAGGAGGTTT CACTGGAAGA | 1950 | |
| GAGGCTGGGC TTGAGTGAGA AGTGAGACAC ACGCGAGTTT CCGGTGAACT | 2000 | |
| CGGCACCAAA CACCTCAGTT TGCTGCTCAG ATGAGGTGTC AGAAAATGCT | 2050 | |
| (SEQ ID NO:1) | | |
| GGGGCTG CACAGCAGGG AGAGTGCTGT GTTATGCGAG | | var (4135):[A:0.14] |
| GAGGTTGGAG AAATCCTCCC CATGAGATAA GATGG/AGAACA GAGATCGGGA | 4150 | |
| CGAAACAAGG GGAGGGGACA | | |
| (SEQ ID NO:2) | | |

The bolded numbers to the right indicate the nucleotide number within the IL4-IL13 haplotype block. The bolded letters indicate the nucleotide variation. The bracketed numbers indicate the frequency of the variation.

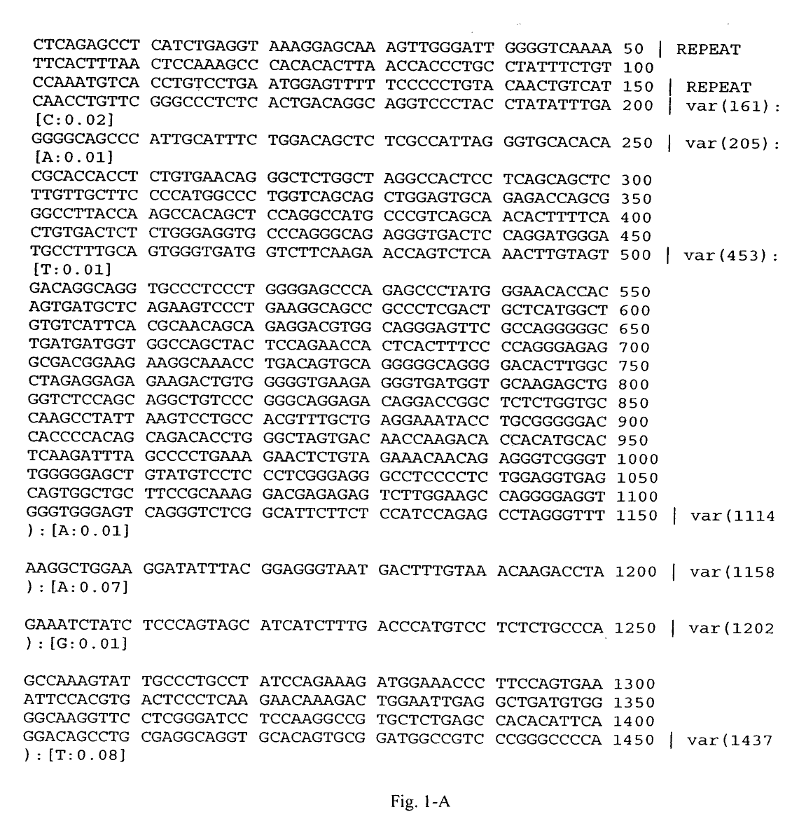

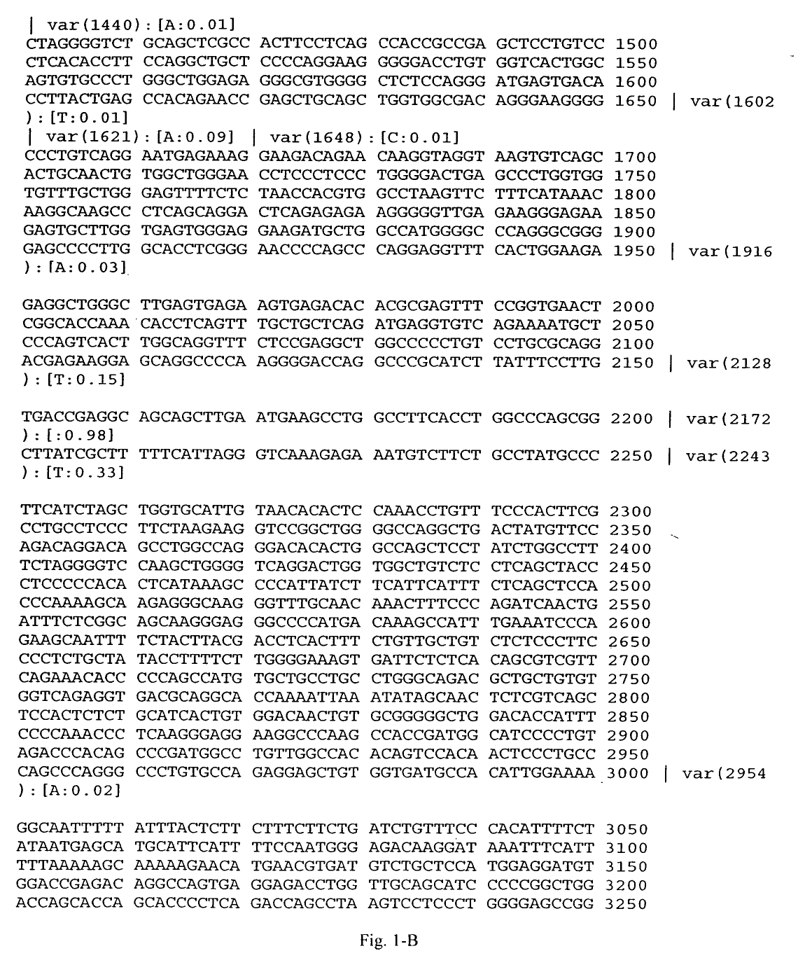

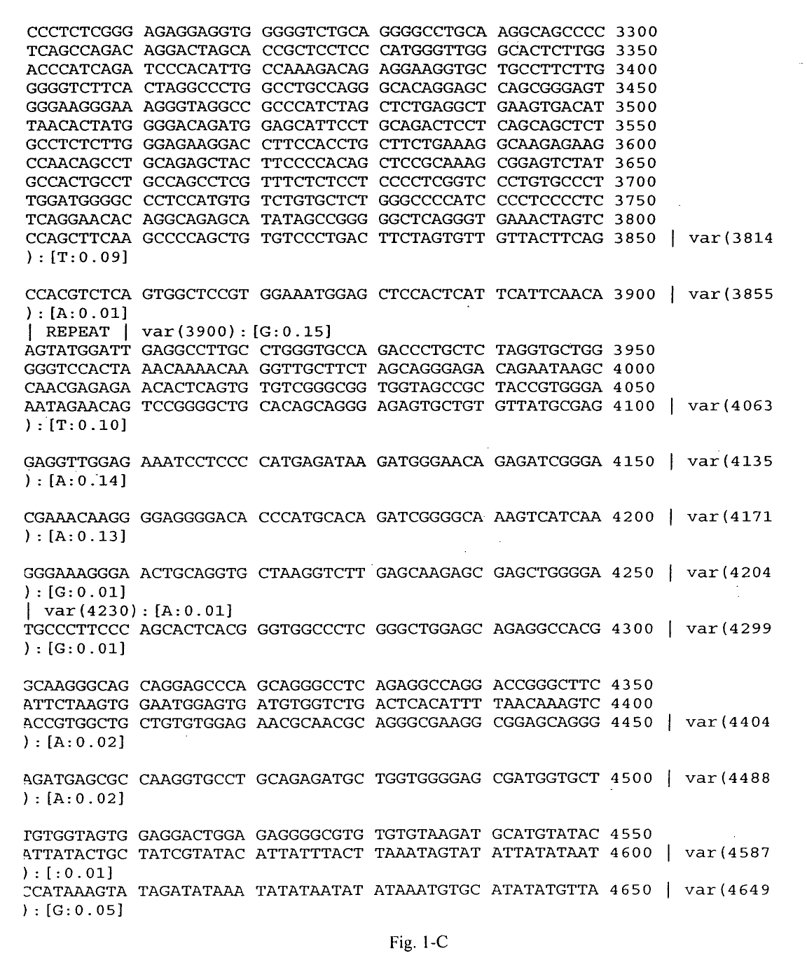

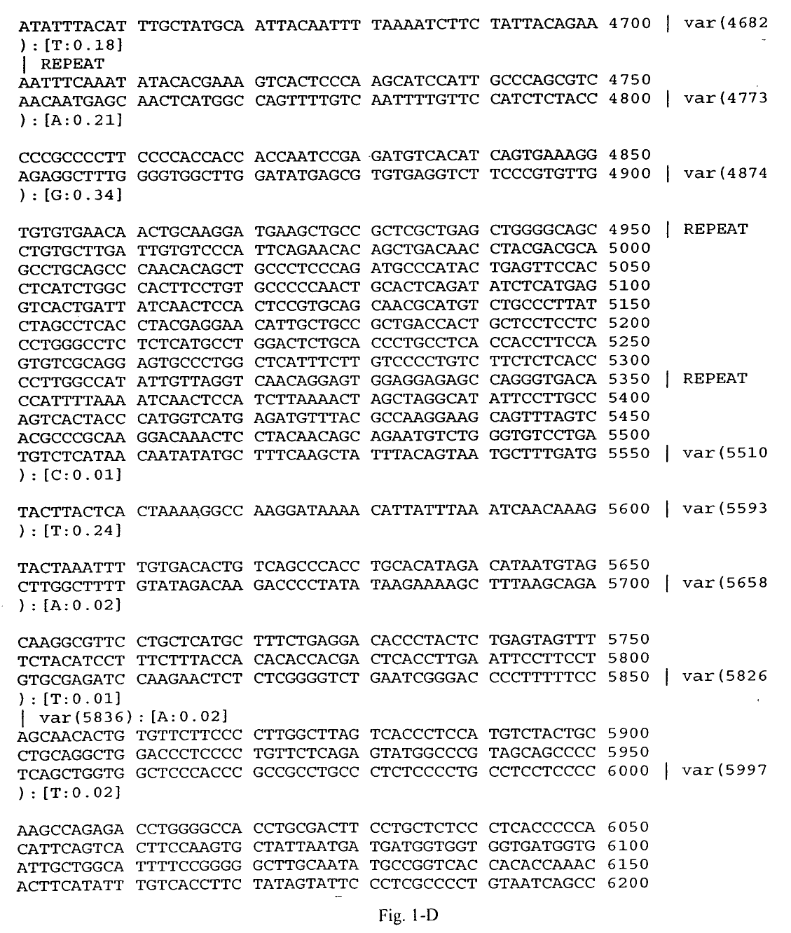

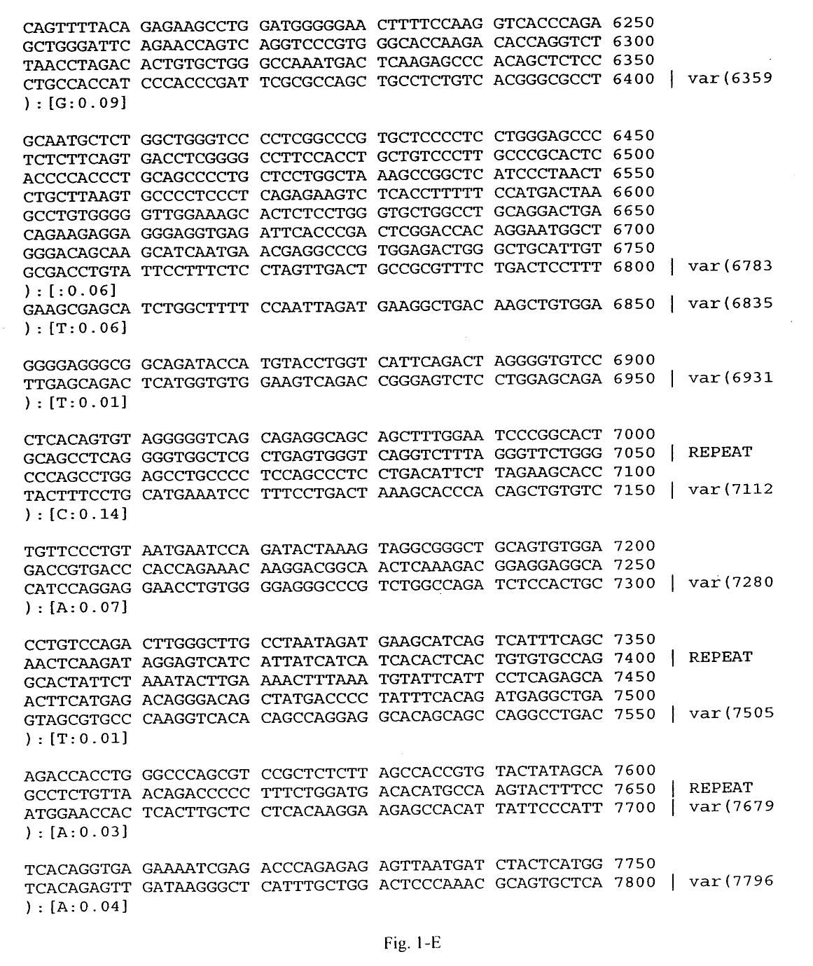

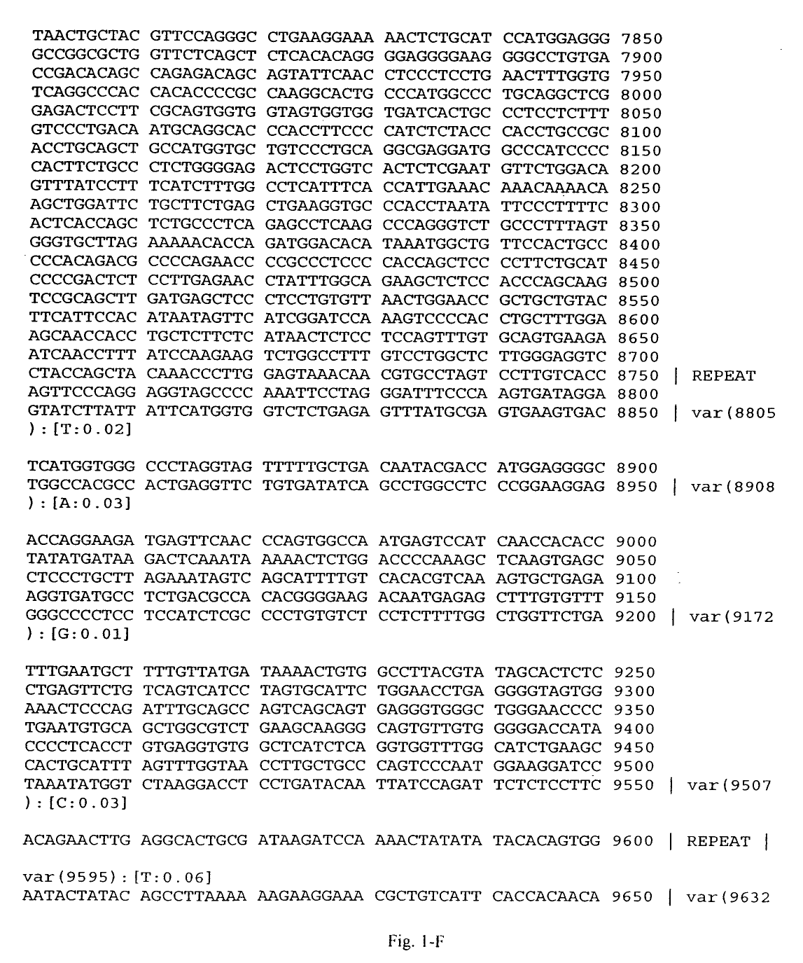

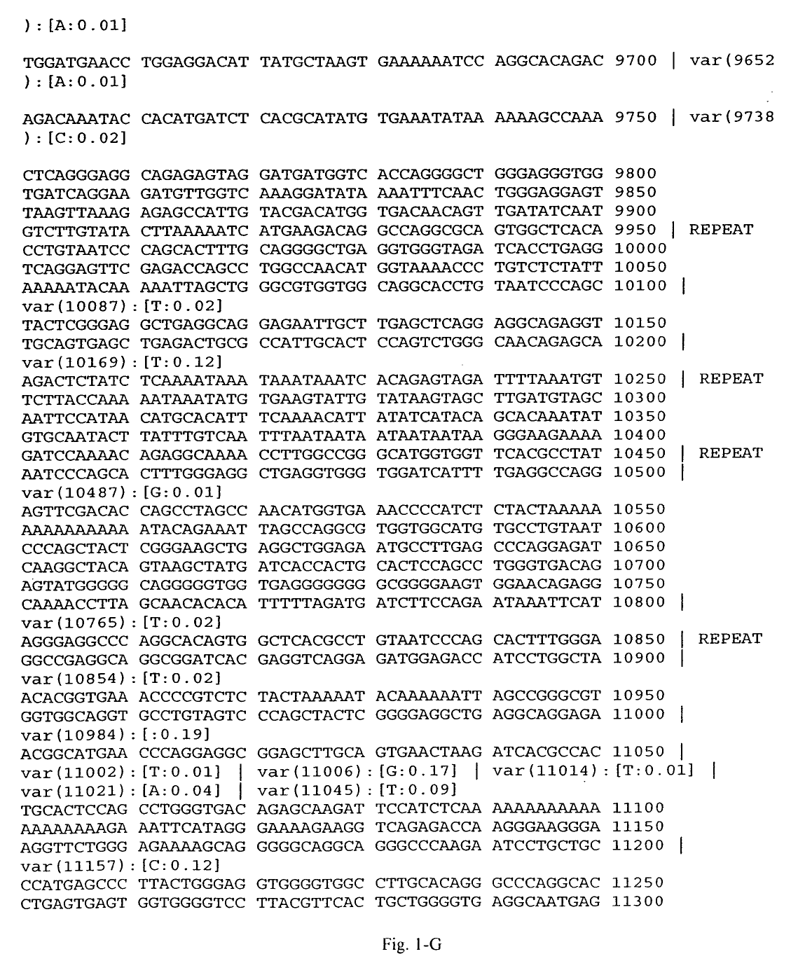

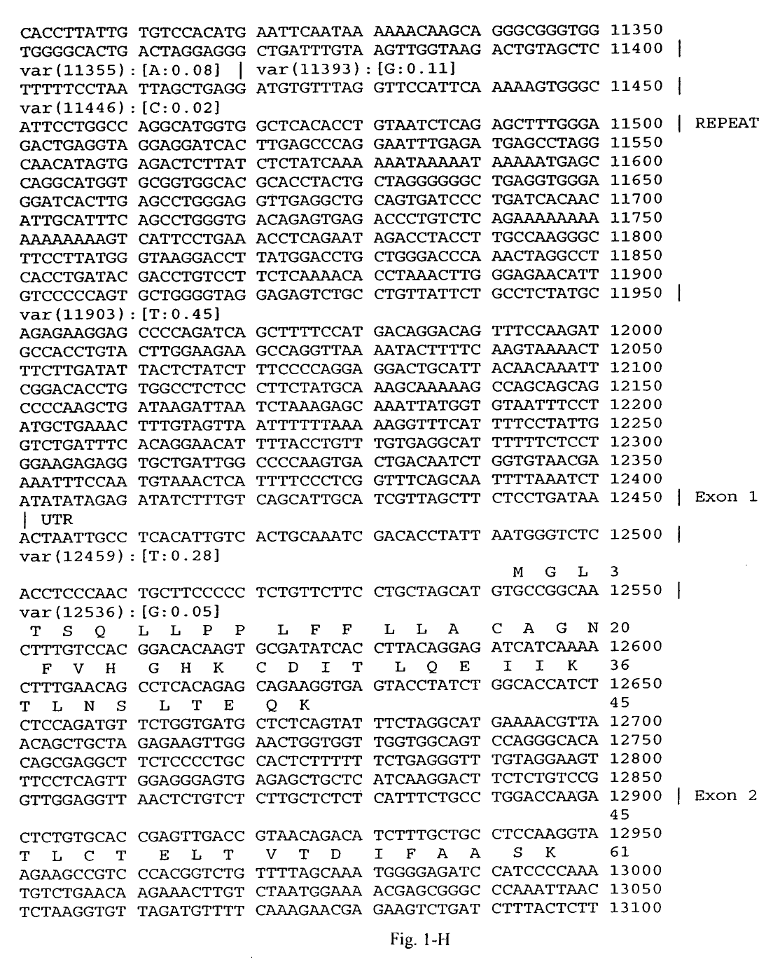

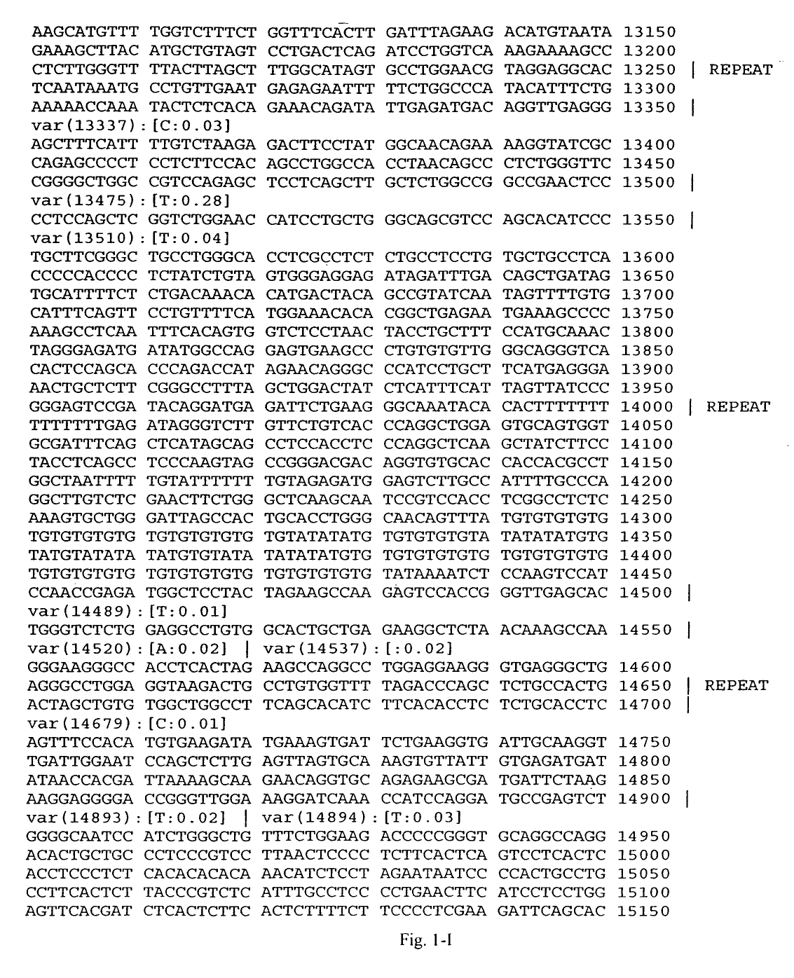

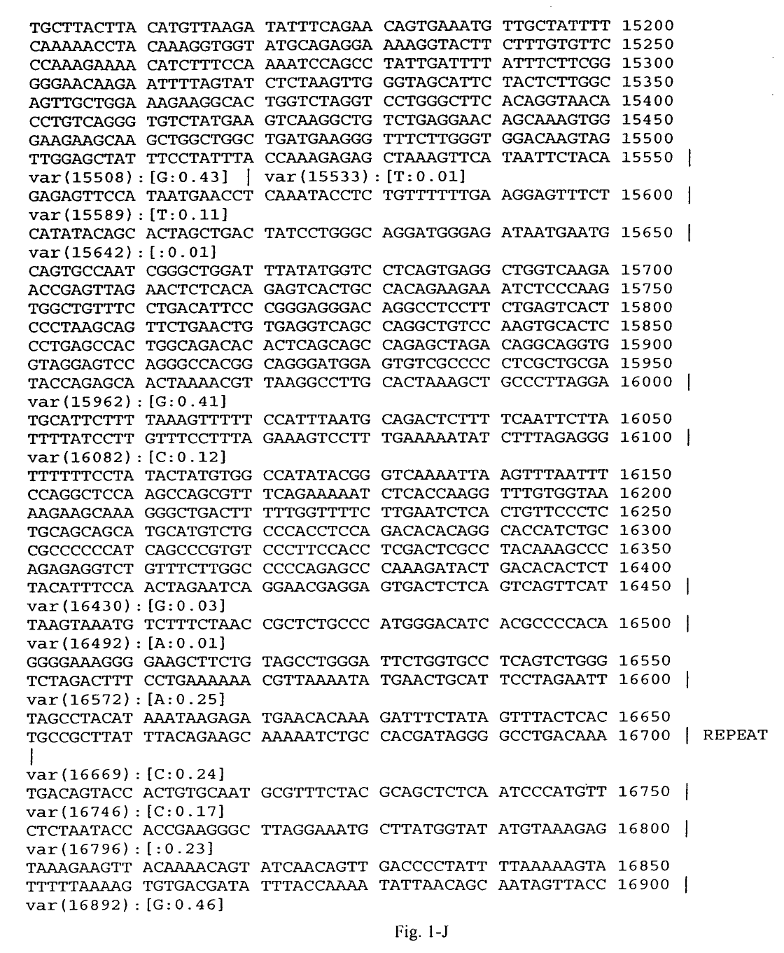

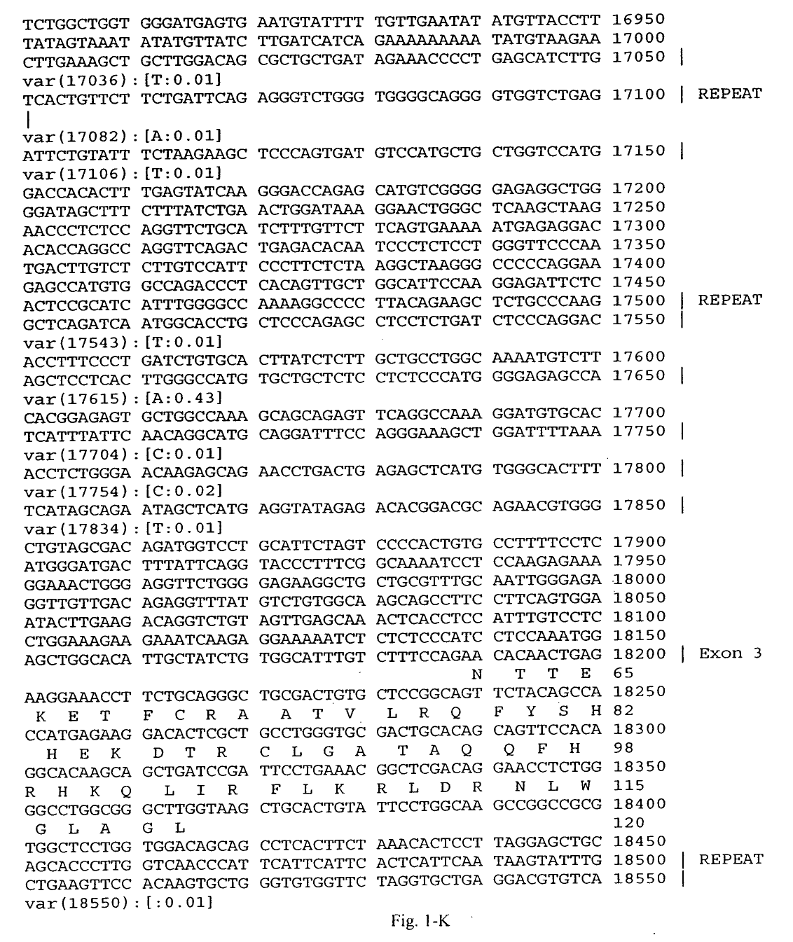

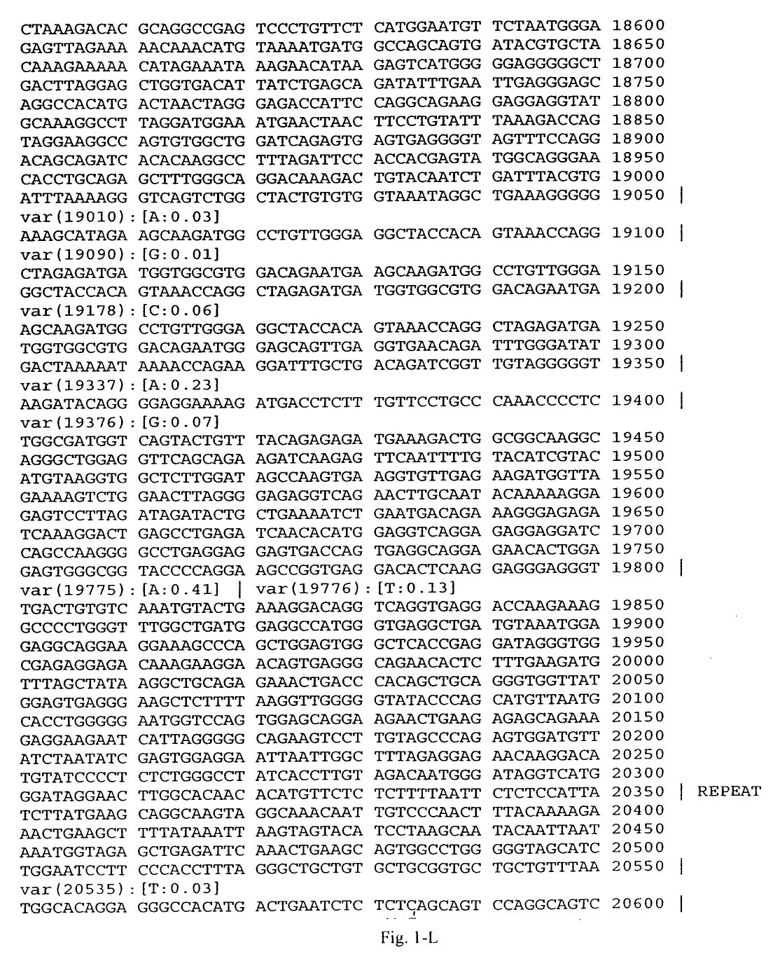

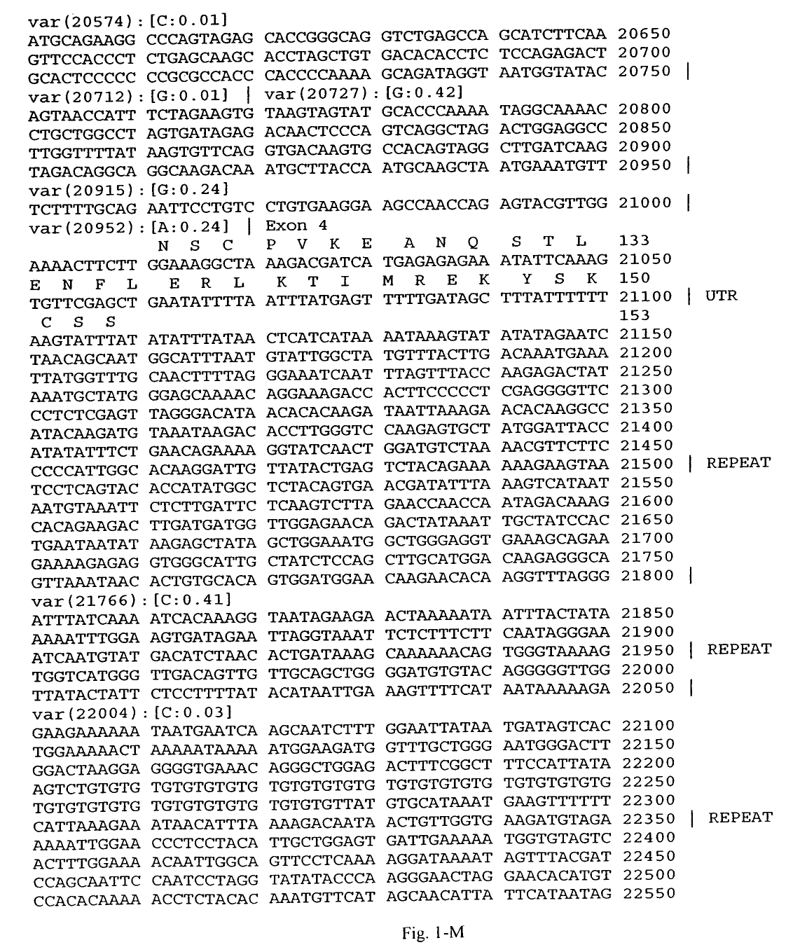

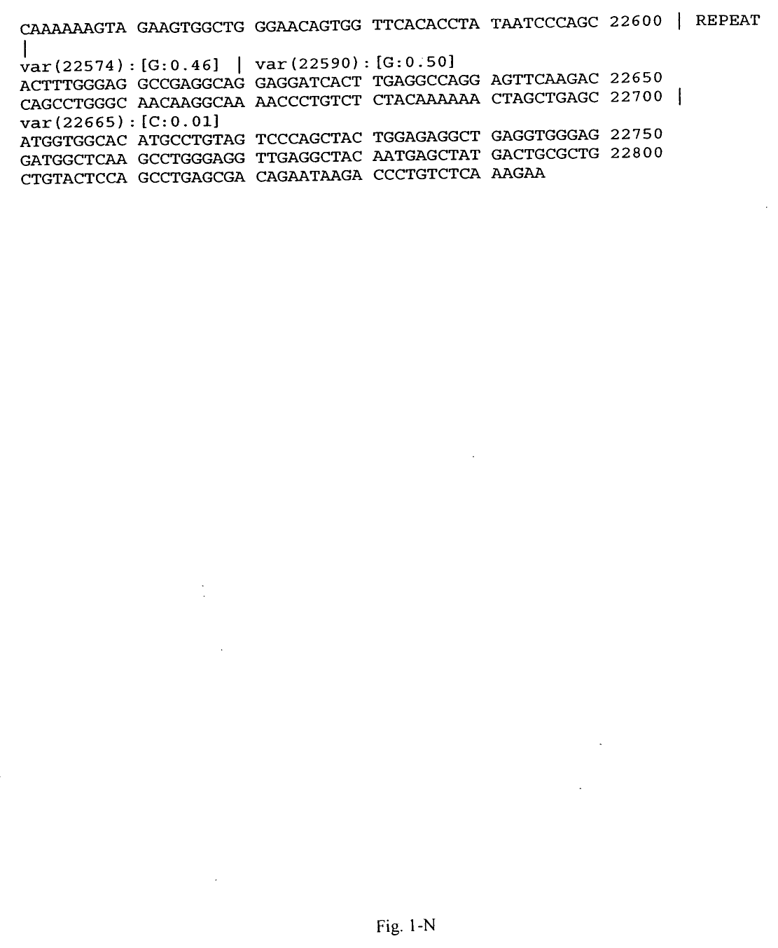

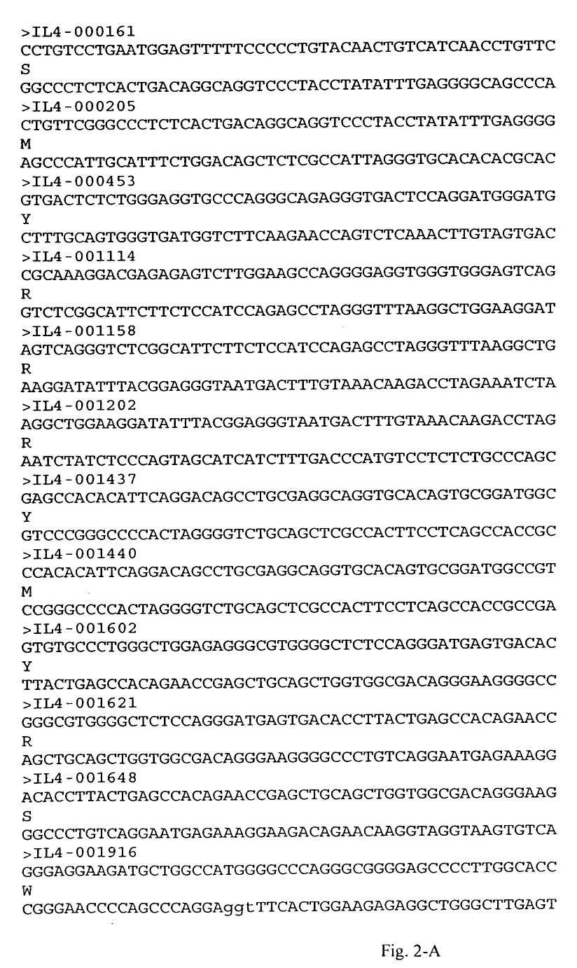

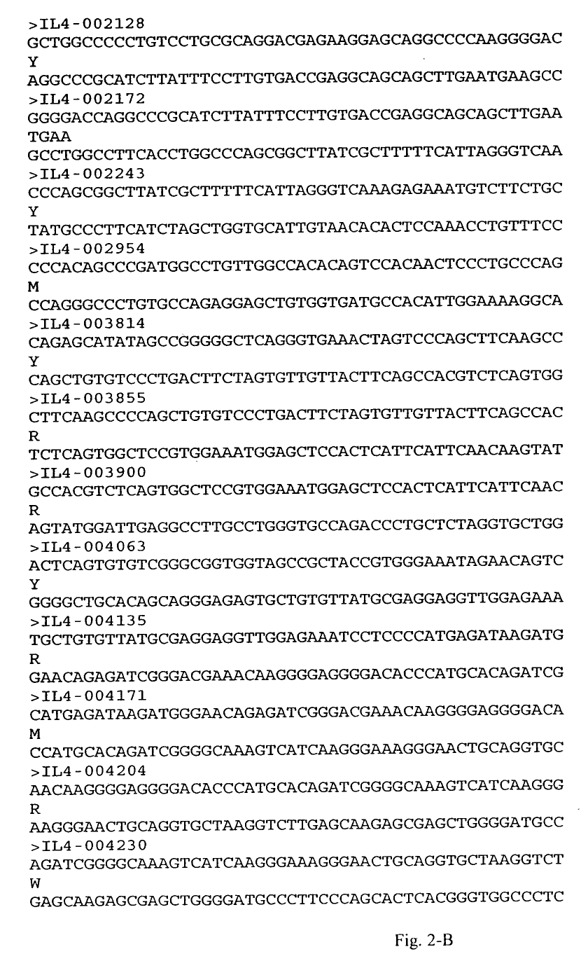

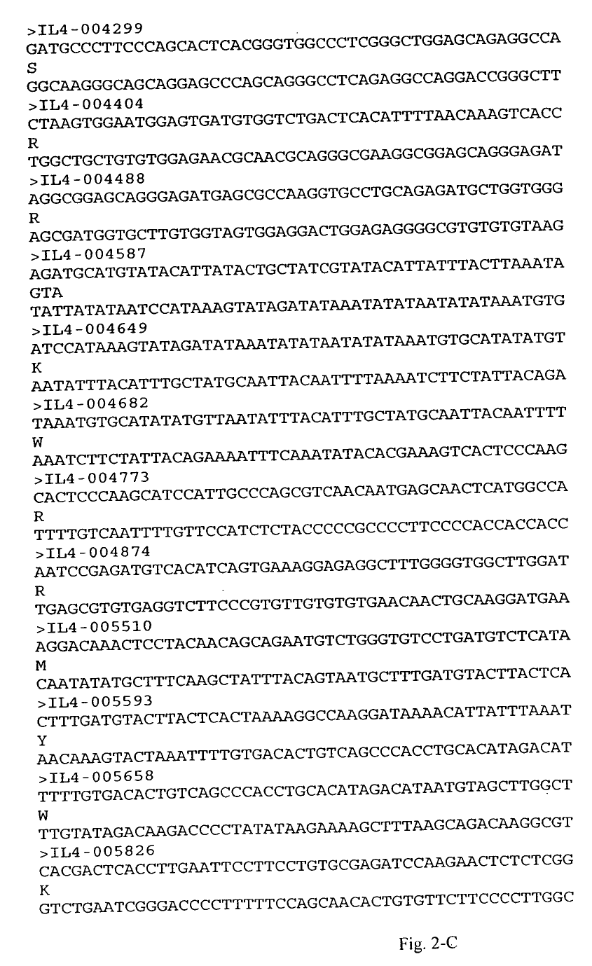

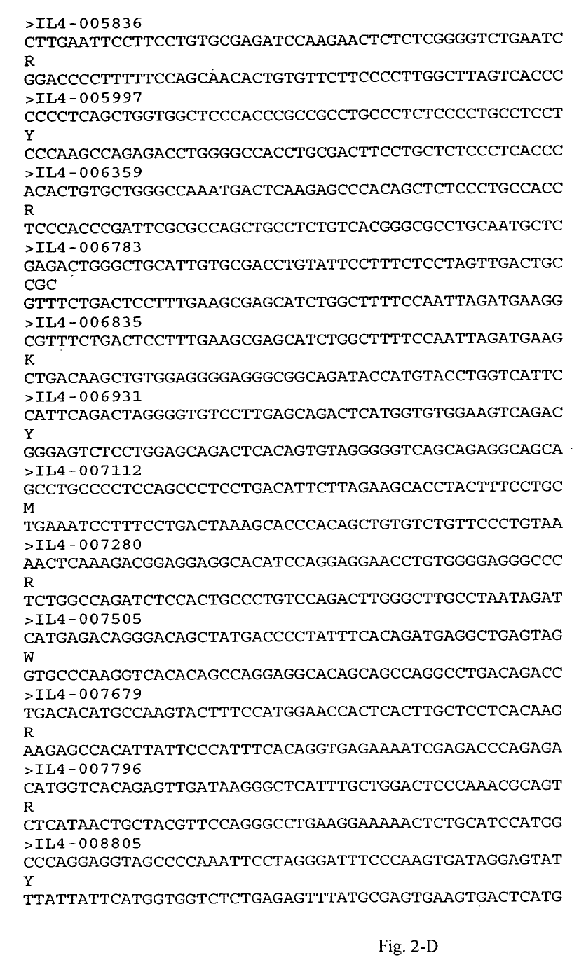

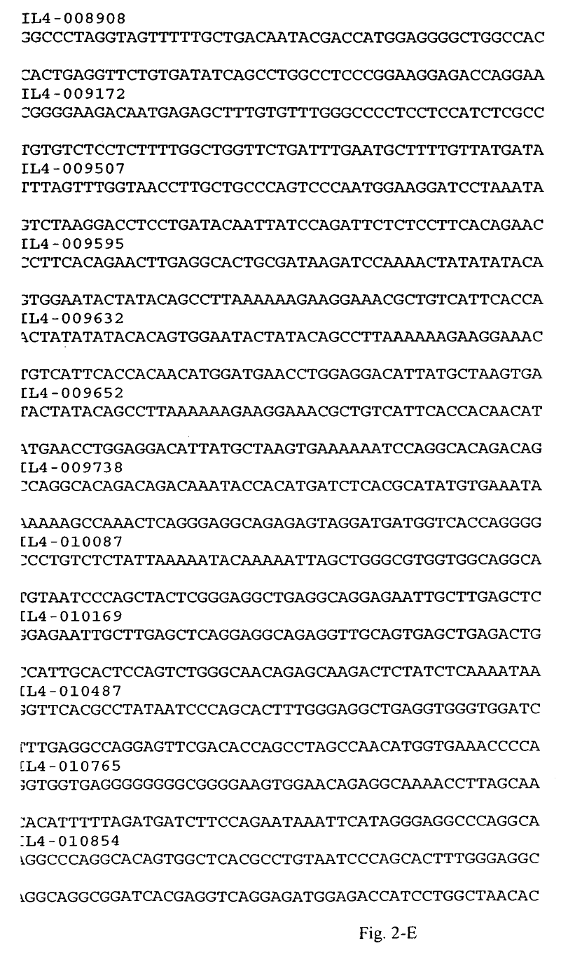

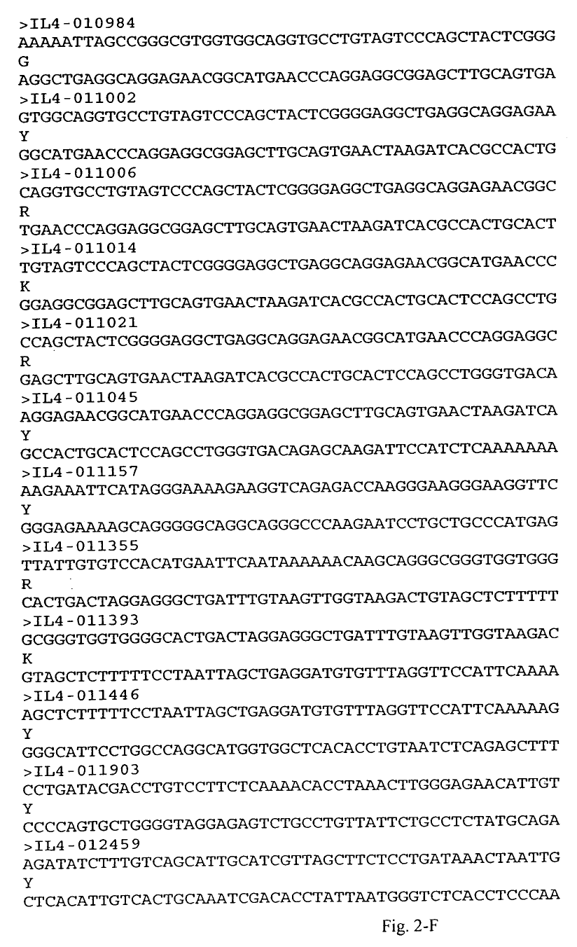

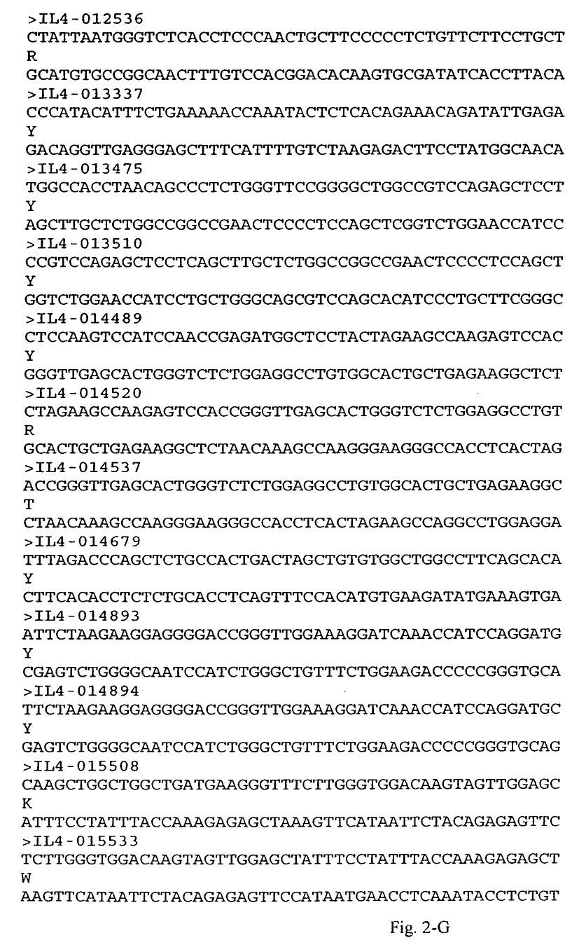

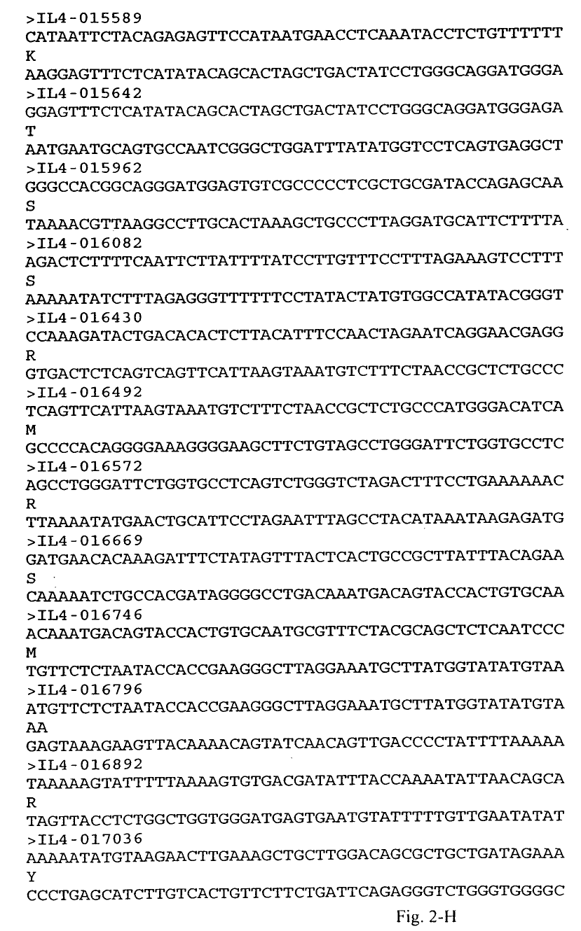

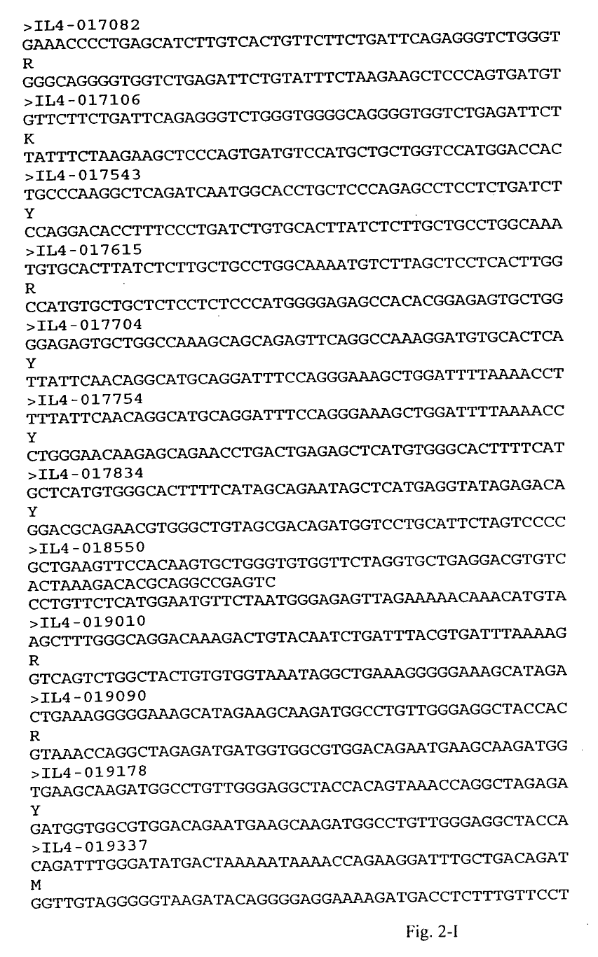

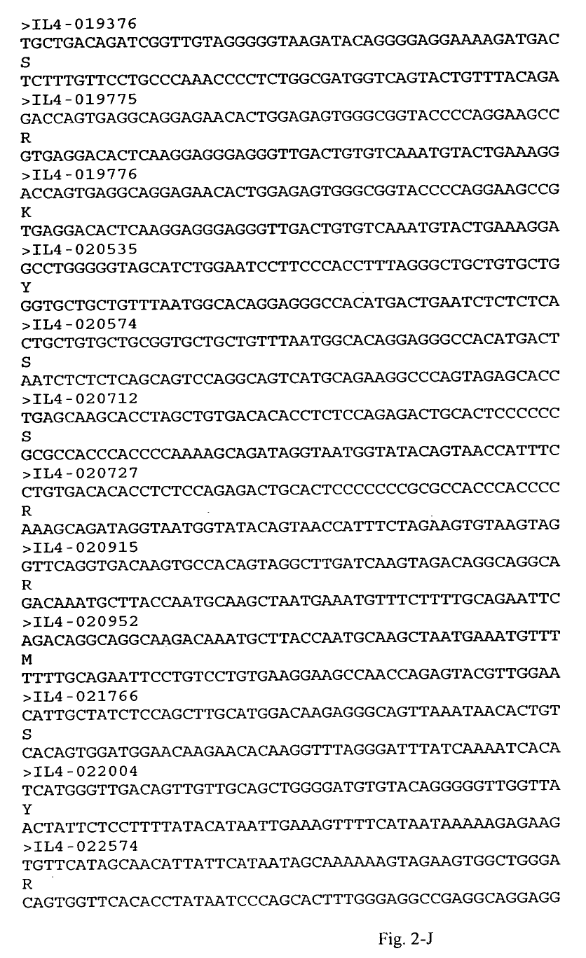

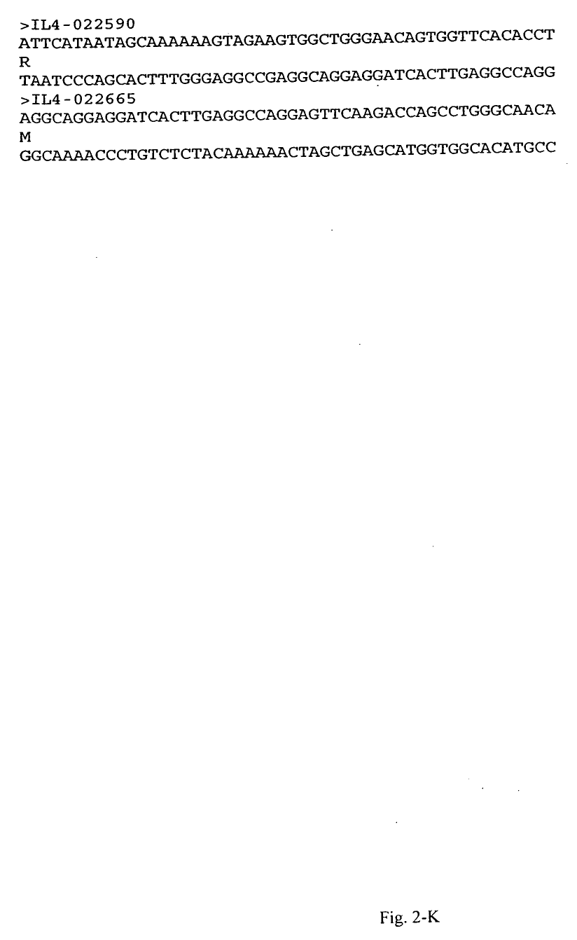

FIGS. 1a-1n and 2a-2k are supplied to further illustrate the SNPs. FIGS. 1a-1n (SEQ ID NO:3) show the annotated DNA sequence for the IL4 region with SNPs. FIGS. 2a-ak (SEQ ID NO:4) show SNPs in the IL4 region with flanking sequences, including a detailed description of the base pair changes using the IUPAC letters (i.e., R=A or G, Y=C or T, M=C or A, K=T or G, W=T or A, and S=G or C).

We also demonstrate an interaction between IL4_{—}4135 and SNP CRP_{—}2667 in the CRP gene. In particular, individuals who are homozygous for the common allele of both IL4_{—}4135 and CRP_{—}2667 have the highest mean serum level of CRP while those who are homozygous for the minor allele of both have the lowest levels. Individuals who are heterozygous for one and homozygous for the common allele of the other of IL4_{—}4135 and CRP_{—}2667 have intermediate serum CRP levels. SNP CRP_{—}2667 is known in the art. For example, it is known that the common allele of CRP_{—}2667 has a “G” at position 2667 of the CRP gene and the minor allele has a “C” at that position.

We further demonstrate here that individuals who carry the minor allele of IL4_{—}1916 develop myocardial infarction (MI) for the first time at an older average age than individuals who do not carry the minor allele of IL4_{—}1916. By the minor allele of IL4_{—}1916, we mean that the nucleotide at nucleotide position 1916 of the IL4-IL13 intergenic region of human chromosome 5 is “A” (Table 1 and FIGS. 1a-1n and 2a-2k). The corresponding position for the common allele of IL4_{—}1916 has a “T” (Table 1 and FIGS. 1a-1n and 2a-2k).

IL4_{—}4135 and IL4_{—}1916 are located in transcription factor binding sites and we demonstrate the functional importance of these two SNPs using in vitro transfection assays which show that the two SNPs manifest allele-specific transcription factor binding site activities.

In one aspect, the present invention relates to a method of correlating a human subject's serum or plasma CRP level to the subject's genetic composition. The method involves (a) determining the status of a marker selected from single nucleotide polymorphism (SNP) marker IL4_{—}4135 or another SNP marker in linkage disequilibrium with IL4_{—}4135 in the genome of the human subject and (b) correlating the result from step (a) to the subject's serum or plasma CRP level wherein on average subjects who carry the minor allele of IL4_{—}4135 have a lower serum or plasma CRP level than subjects who do not carry the minor allele of IL4_{—}4135. The serum or plasma CRP level of the subject can be provided or measured before, at the same time, or after the status of said SNP marker or markers are determined. By “determining the status of a SNP,” we mean one or more of the following: (i) determining whether an individual carries an allele (e.g., a minor or the common allele) of interest of the SNP, (ii) determining whether an individual is heterozygous or homozygous for a specific allele of interest of the SNP (i.e., genotyping), and (iii) determining which the specific allele or alleles of the SNP that an individual carries.

In another aspect, the present invention relates to a method of screening a human subject for propensity to develop a cardiovascular disease. The method involves (a) determining the status of a marker selected from single nucleotide polymorphism (SNP) marker IL4_{—}4135 or another SNP marker in linkage disequilibrium with IL4_{—}4135 in the genome of the human subject; and (b) correlating the result from step (a) to the subject's propensity for developing the cardiovascular disease wherein subjects carrying the minor allele of IL4_{—}4135 are less likely to develop the cardiovascular disease than subjects who do not carry the minor allele of IL4_{—}4135.

In another aspect, the present invention relates to a method of correlating a human subject's serum or plasma CRP level to the subject's genetic composition. The method involves (a) genotyping the genome of the human subject for SNP marker IL4_{—}4135 or another SNP marker that is in linkage disequilibrium with IL4_{—}4135, (b) genotyping the genome of the human subject for SNP marker CRP_{—}2667 or another SNP marker that is in linkage disequilibrium with CRP_{—}2667, and (c) correlating the result from step (a) to the subject's serum or plasma CRP level wherein on average individuals who are homozygous for the common allele of both IL4_{—}4135 and CRP_{—}2667 have a higher serum or plasma CRP level than individuals who are heterozygous for one and homozygous for the other of IL4_{—}4135 and CRP_{—}2667, who are in turn have a higher serum or plasma CRP level than individuals who are homozygous for the minor allele of both IL4_{—}4135 and CRP_{—}2667.

In another aspect, the present invention relates to a method of screening a human subject for propensity to develop a cardiovascular disease. The method involves (a) genotyping the genome of the human subject for SNP marker IL4_{—}4135 or another SNP marker that is in linkage disequilibrium with IL4_{—}4135, (b) genotyping the genome of the human subject for SNP marker CRP_{—}2667 or another SNP marker that is in linkage disequilibrium with CRP_{—}2667, and (c) correlating the result from step (a) to the subject's propensity for developing the cardiovascular disease wherein individuals who are homozygous for the common allele of both IL4_{—}4135 and CRP_{—}2667 are more likely to develop the cardiovascular disease than individuals who are heterozygous for one and homozygous for the other of IL4_{—}4135 and CRP_{—}2667, who are in turn more likely to develop the cardiovascular disease than individuals who are homozygous for the minor allele of both IL4_{—}4135 and CRP_{—}2667. The serum or plasma CRP level of the subject can be provided or measured before, at the same time, or after the status of said SNP markers are genotyped.

In still another aspect, the present invention relates to a method of screening a human subject for predicting time to first myocardial infarction (MI). The method involves (a) determining the status of a marker selected from SNP marker IL4_{—}1916 or another SNP marker in linkage disequilibrium with IL4_{—}1916 in the genome of the human subject, and (b) correlating the result from step (a) to time to first myocardial infarction wherein subjects carrying the minor allele of IL4_{—}1916 are likely to have myocardial infarction for the first time at an older age than subjects who do not carry the minor allele of IL4_{—}1916.

There are many methods to analyze SNPs. For example, one can obtain a nucleic acid sample such as a DNA sample for an individual and determine the presence of the SNPs with any the following methods: The SNPs can be assayed using standard DNA sequencing technology such as ABI dye terminator chemistry on an ABI sequencer. The SNPs are also amenable to detection using any standard SNP genotyping platform such as TaqMan (ABI), mass spectroscopy (Sequenome), various single base pair extension assays, and chip based genotyping platforms (Affymetrix) or bead array platforms (Ilumina).

When we indicate that one would determine the status of a SNP, we mean that one could determine the status of the SNP directly by examining the nucleotide position of the SNP itself or indirectly by determining the status of one or more markers (e.g., SNP markers) that are in linkage disequilibrium with the SNP.

One of skill in the art would understand that there are many ways to evaluate the linkage between two or more markers such as SNPs. Suitable metrics are described in Hedrick, P.W., Genetics 117(2):331-341,1987. For the purposes of the present invention, markers are in a suitable linkage disequilibrium if D′ is greater than 0.8.

The invention will be more fully understood upon consideration of the following non-limiting example.

### EXAMPLE

### Functional Polymorphisms in the IL4-IL13 Intergenic Region Influence CRP Levels and Time to First MI

In this example, we demonstrate the positional identification of genetic variation within the IL4-IL13 intergenic region influencing serum CRP levels. Using linkage analysis followed by positional candidate gene association analysis, we identified five single nucleotide polymorphisms within a single haplotype block in the IL4-IL13 intergenic region, being associated with CRP levels in a large myocardial infarction family-set. We further demonstrate a joint effect on high sensitivity serum CRP (hsCRP) levels between SNP IL4_{—}4135 and previously identified SNPs in the CRP gene. Both the association and the combined effect of both polymorphisms were replicated in a second independent population-based study sample. In addition, we demonstrate that the identified SNPs attenuate our initial CRP linkage signal, indicating that these polymorphisms are the underlying correlate of the initial QTL. We also demonstrate that these SNPs alter reporter-gene expression levels. Lastly, we demonstrate that one of the SNPs, together with other established risk factors, significantly affects the age of first MI in the MI family set. While the studies presented here were conducted with Caucasian families, it is expected that the observations are applicable to other populations such as the human population in general given that the functions of CRP and its connection to cardiovascular diseases are conserved across the human population in general.

Methods

A. Subjects and Phenotyping.

All study participants gave written informed consent and the study was approved by the ethics committee at the Medical College of Wisconsin and University of Regensburg, Germany.

B. MI Family Set.

Subject ascertainment and phenotyping, MI/CAD: An in-depth description of the patient ascertainment strategy and the clinical characteristics of the study population have been described in (Broeckel U, et al., Nat Genet 2002;30(2):210-4). Briefly, Western European families were included in the study if probands had suffered from an MI (as documented by criteria chosen according to the published definitions of the MONICA (Monitoring Trends and Determinants in Cardiovascular Disease) investigators of the World Health Organization (WHO)) before the age of 60 and affected siblings had an MI or had undergone percutaneous transluminal coronary angioplasty (PTCA) or bypass grafting (CABG). Time to the first MI was determined based on medical records.

High sensitivity serum CRP (hsCRP) measurements: Blood was obtained from individuals without signs of overt infection or unstable angina. The time between an acute MI and blood collection was at least 2 months. Serum was stored at −80° C. and thawed only once for analysis. hsCRP was quantified by means of particle-enhanced immunonephelometry according to the instructions of the manufacturer (Dade Behring Nephelometer Systems). The lower limit of detection was 0.2 mg/L. The intra- and inter-assay variabilities were 3.1 and 2.5%.

TABLE 2
Clinical Characteristics of MI Family Data-set.

| | | | Unaffected |
|---|---|---|---|
| | MI Patients | Affected Sibs | Sibs |
| | N = 513 | N = 618 | N = 238 |
| Characteristic | (μ ± SD) | (μ ± SD) | (μ ± SD) |
| Age at Ascertainment | 60.0 ± 0.4 | 62.4 ± 0.3 | 59.6 ± 0.5 |
| MI/PTCA/CABG (N) | 513/216/267 | 408/235/353 | — |
| Age at First MI | 51.6 ± 0.4 | 55.1 ± 0.5 | — |
| Gender (% male) | 80.9 | 78.6 | 41.8 |
| InCRP Level | 0.61 ± 1.2 | 0.79 ± 1.18 | 0.63 ± 1.13 |
| Diabetes Mellitus (%) | 14.4 | 16.3 | 8.4 |
| BMI | 27.1 ± 0.2 | 27.2 ± 0.1 | 27.2 ± 0.2 |
| Cigarette Smoking | 73.1 | 70.2 | 48.9 |
| (%) | | | |
| Systolic BP (mmHg) | 141 ± 1 | 144 ± 1 | 148 ± 1 |
| Diastolic BR (mmHg) | 83 ± 1 | 84 ± 1 | 86 ± 1 |
| Total Cholesterol | 228 ± 2 | 235 ± 2 | 256 ± 3 |
| (mg dl⁻¹) | | | |
| LDL Cholesterol | 159 ± 2 | 163 ± 2 | 183 ± 3 |
| (mg dl⁻¹) | | | |
| HDL Cholesterol | 48 ± 1 | 50 ± 1 | 54 ± 1 |
| (mg dl⁻¹) | | | |
| Lipid-lowering | 57.7 | 53.8 | 12.2 |
| Medication (%) | | | |
| Anti-hypertensive | 82.8 | 82.1 | 42.6 |
| Medication (%) | | | |

C. Genome Scan and Linkage Analysis.

DNA was extracted from peripheral blood lymphocytes (Gentra Puregene DNA extraction kit). Genotyping for the genome scan analysis was performed by the Mammalian Genotyping Service, Marshfield Medical Clinic with a total of 394 microsatellite markers covering all autosomal chromosomes with an average distance of 10 cM (screening set 10). Variance component linkage analysis was performed using the SOLAR genetic analysis program (Almasy L, et al., Am J Hum Genet 1998;62(5):1198-211). The initial analyses screened for general covariables including age, gender, diabetes, cigarette smoking, BMI, LDL, systolic and diastolic hypertension, MI, aspirin use, statin use, blood pressure medication, and hormone replacement therapy. Only significant covariables (p<0.1) were retained for the subsequent linkage analysis. Diabetes mellitus was defined by the use of anti-diabetic medication (oral anti-diabetics or insulin injections) or by a concentration of glycosylated hemoglobin (HbA1c) ≧6.5%. Smoking was categorized as either current/former smoker or never smoker at the time of MI. We defined systolic hypertension as systolic blood pressure ≧140 mmHg and diastolic hypertension as ≧90 mmHg irrespective of the intake of antihypertensive medication. This conservative approach in defining hypertension reduces the probability that an individual is falsely categorized as hypertensive. To account partially for the non-random sampling, we conditioned the likelihood of a family on the MI phenotype of the initial proband. P-values were estimated empirically by using simulation methods incorporated into SOLAR. The heritability of hsCRP (high sensitivity serum CRP) was estimated at 0.31 ±0.07 (p=0.0000015) under a model adjusted for the significant covariables age, gender, diabetes, body mass index (BMI), and smoking.

D. SNP Identification and Genotyping.

Haplotype-tagging SNPs with a minor allele frequency (MAF) ≧5% for each of the candidate genes were selected based on the CEPH data from the SeattleSNPs Program for Genomic Applications (SeattleSNPs. NHLBI Program for Genomic Applications, UW-FHCRC, Seattle, Wash. (URL: http:/lpga.gs. washington.edu). The tested genes included IL-3, IL-4, IL-5, IL-9, IL12B, IL-13, IL17B, CRP, and Colony Stimulating Factor-2 (CSF2).) SeattleSNPs uses a unique algorithm to determine haplotype-tagging SNPs within a gene (Carlson CS, et al., Nat Genet 2003;33(4):518-21). Briefly, haplotype-tagging SNPs are determined based on a binning algorithm which identifies the single SNP that exceeds a threshold level of linkage disequilibrium (as determined by the measure of r²) with the maximum number of other SNPs. This group of SNPs is set as a bin. Each SNP within a bin is then analyzed to determine whether it exceeds the threshold level of LD with all other SNPs in that bin. All SNPs within a bin that meet this criterion are designated as TagSNPs. SNPs are referred to by their sequence coordinate based on SeattleSNPs' sequencing data. Genotyping was performed using the Applied Biosystems' (ABI, Foster City, Calif.) TaqMan technology. Initial SNPs within each of the five conserved non-coding sequence (CNS) regions, as described by Loots et al. (Science 2000;288(5463):136-40), were identified in 24 unrelated Caucasian individuals (12 cases with MI and 12 controls without MI) from the German population using direct fluorescence-based sequencing incorporating Big Dye Terminator chemistry (ABI, Foster City, Calif.). A Polyphred quality score of 20 was used in SNP identification. Identified SNPs with a MAF≧5% were then genotyped in the families using the TaqMan platform.

E. Statistical Analysis.

Linkage Disequilibrium Estimation: The analysis program Haploview (version 3.11) was used to calculate and visualize linkage disequilibrium and haplotype-block patterns of the genotyped SNPs (Barrett J C, et al., Bioinformatics 2005;21 (2):263-5). Haplotype blocks were determined based on the confidence interval method, as implemented in Haploview.

Association Analysis: hsCRP levels were log transformed to account for the non-normal distribution. The QTDT program was used for the quantitative family-based single SNP association analysis (Abecasis G, et al., Am J Hum Genet 2000;66:279-92). The initial association analysis was performed in a subset of the families which contributed to the LOD score based on the calculation of family-specific LOD scores for the peak position of the initial linkage signal as implemented in the genetic analysis software package SOLAR (Almasy L, et al., Am J Hum Genet 1998;62(5):1198-211). SNPs which showed a significant association (p<0.05) were subsequently typed in the full family set. P-values for single SNP association analysis were adjusted for age, gender, body-mass index (BMI), presence or absence of diabetes, smoking status, presence or absence of CAD/MI, lipid medication, total cholesterol:HDL ratio, and the three SNPs within the CRP gene demonstrating significant associations with CRP levels (CRP-2667, CRP-3872, CRP-6192). The PBAT software with offsets determined by genetic effect size was used to examine interaction between the chr. 5 IL4-IL1 3 SNPs and the chr. 1 CRP SNPs (Lange C, et al., Am J Hum Genet 2004;74(2):367-9). The difference in mean InCRP levels among the different genotypic categories of the IL4-IL13 intergenic region SNPs and CRP SNPs was tested with an One-way ANOVA test as implemented in SigmaStat v. 2.03.

Conditional Linkage Analysis/Quantitative Trait Nucleotide Analysis: SNPs demonstrating a positive association with CRP levels in the full dataset were subsequently incorporated into our original linkage model to test if they could account for the initial linkage signal observed on chr. 5 using the measured genotype approach. The measured genotype approach utilizes the variance not accounted for in modeling of genetic effects (QTL effects and residual additive genetic effects) and measured covariates (e.g. age, sex, race etc.) to test whether a particular polymorphism accounts for an observed QTL. We specified an additive model in which the heterozygote mean is intermediate to the two homozygote means. If the measured genotype (e.g. the SNP) is a variant influencing the trait, the IBD allele sharing will provide no additional information and the LOD score will be reduced (Almasy L, et al., Behav Genet 2004;34(2):173-7). These analyses were performed using the program package SOLAR, in which the SNP genotypes were added as covariates to the original linkage model.

Cox-Proportional Hazards Modeling: In order to evaluate the influence of the significant SNPs on time to first MI, Cox proportional hazard regression models were performed with adjustments for possible dependencies within families using a frailty model. In a frailty model, each member of the family shares a common unobservable random effect that modifies their hazard rate by a common multiplicative factor and this random effect was modeled by a gamma distribution (Hougaard P. New York: Springer-Verlag; 2000; Neale MC, et al., Dordrecht: Kluwer Academic Press; 1992).

Our approach was to fit a series of regression models, each with an appropriate frailty. For each possible variable to be entered in the regression model, we determined proportional hazards and checked model fit by using appropriate residual plots (Klein JP, et al., New York: Springer-Verlag; 1997).

The first set of models includes the identified risk factors measured on the subject (e.g., gender, smoking history, diabetes history, etc.), which were adjusted for in subsequent models. Next, we fitted models that included CRP information with indicators of the possible genetic predictors of CRP. At each step we examined not only the significance of the risk coefficients, but also the strength of association between family members.

Variables with p-values of less than 0.05 were considered statistically significant. Analysis was carried out with SAS version 9 and S-PLUS version 7.0 statistical software.

F. Functional Analyses of IL4-IL13 Intergenic Region SNPS.

Promoter-Variant Vector Construction: PCR primers were designed with unique SacI (forward) and KpnI/XhoI (reverse) sites up and downstream of the SNPs to be amplified. Following amplification, PCR products of approximately 300-1000bp 5′ and 3′ of each SNP were cloned into SacI/KpnI/XhoI digested pGL3-promoter and enhancer luciferase vectors (Promega, Madison, Wis.). All constructs were sequence-verified for the presence of each allele respectively using direct fluorescence-based sequencing on an ABI 3730 sequencer.

Cell Culture: HepG2 cells were grown in 0.1 micron- filtered DMEM including high glucose, L-glutamine, pyridoxine hydrochloride and no sodium pyruvate. 15% FBS, 1% L-glutamine, and 1% antimycotic/antibiotic were added and filter sterilized. Cells were grown to 90-95% confluency in 250 mL polystyrene Falcon culture flasks. To split, cells were washed with 1× Phosphate Buffered Solution and then incubated at 37° C. in 0.25% Trypsin solution for 2-5 minutes. Serum-containing growth medium was then added and cells were split into appropriate containers. Jurkat T-cells were grown in 75cm² plug seal culture flask (Fisher Scientific) with RPMI media (Invitrogen, Carlsbad, Calif.) supplemented with 10% FBS, 1% antibiotic-antimycotic, 1% Sodium Pyruvate, and 1% 1 M HEPES buffer filter sterilized. Cells were split at 80-95% confluency.

Transient transfection luciferase assays: HepG2 cells were seeded in 12-well polystyrene culture plates to 90-95% confluency. Jurkat T-cells were seeded in 12-well plates at a concentration of 1.2×10⁶/mL in 1 mL of cell growth media containing serum and antibiotics. HepG2 cells were transfected with Lipofectamine 2000 (Invitrogen, Carlsbad, Calif.) at a ratio of 1:3 DNA to transfection reagent with equal amounts of pGL3 experimental and phRG-TK renilla plasmids (to control for transfection efficiency). Jurkat T-cells were transfected following the Qiagen Superfect protocol using a DNA to transfection reagent ratio of 1:4 with equal amounts of experimental and renilla plasmids. Cells were incubated for 48 hours (Jurkat T-cells) or 72 hours (HepG2 cells) and then assayed for reporter gene expression using the Dual-Glo Luciferase Assay System (Promega, Madison, Wis.). All experiments were performed each in triplicate in at least two independent determinations.

Results

A. Linkage and Association Analysis.

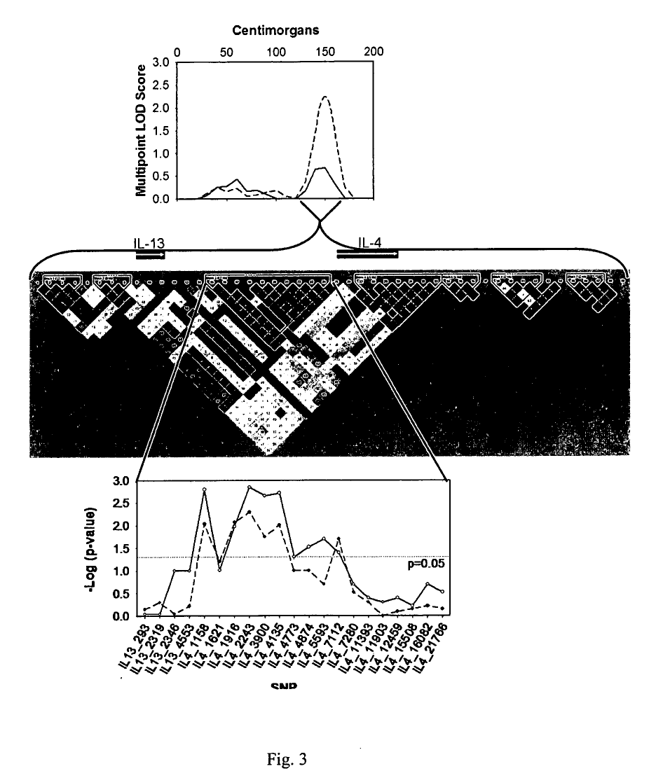

Multipoint linkage analysis identified a region suggestive for linkage on chromosome 5 flanked by the markers D5S1505 and D5S1456 with a peak LOD score of 2.23 (p<0.001) close to D5S1480 (FIG. 3). Following this initial lead, we typed in this linkage region forty haplotype-tagging SNPs with a minor allele frequency (MAF) ≧5% across eight inflammatory candidate genes and five conserved non-coding regions in a subset of 224 families comprised of 712 individuals contributing to the LOD score. Single SNP analysis identified two SNPs located within the IL4-IL13 intergenic region which demonstrated significant associations (p<0.05) with CRP levels. Nine additional markers in strong linkage disequilibrium (LD) with the two positive SNPs were typed, resulting in the identification of three additional significant markers. These five SNPs fall into one LD block comprising half of the IL4-IL13 intergenic region (FIG. 3), including the conserved non-coding sequence element-1 (CNS-1) (Loots GG, et al., Science 2000;288(5463): 136-40).

CNS-1 has previously been shown to act as an enhancer element coordinately regulating interleukins 4, 5, and 13 in mice (Loots GG, et al., Science 2000;288(5463):136-40). In order to identify SNPs within this enhancer element, we re-sequenced CNS-1 in the above subset of contributing individuals (N=712). However, only one rare variant with a minor allele frequency (MAF) of 0.5% was found. Given this extremely low allele frequency, it is unlikely that this SNP plays a functional role within our population.

Subsequent single-locus analysis of the five positive SNPs within the full 513 families revealed significant evidence of association with plasma CRP levels (IL4_{—}1158, p=0.0008; IL4_{—}1916, p=0.0058; IL4_{—}2243, p=0.0015; IL4_{—}3900, p=0.0033; and IL4_{—}4135, p=0.0013). Table 3 summarizes the results of the subset and full dataset single SNP analysis.

TABLE 3
Significant Results (p-values) of Association Analysis in the IL4-IL13
Intergenic Region in a Subset of Informative Families and the Full Dataset
(No. of families) using QTDT.

| | Subset | Full Dataset | Subset | Full Dataset |
|---|---|---|---|---|
| | (N = 224) | (N = 513) | (N = 224) | (N = 513) |
| | Crude Analysis | | *Fully Adjusted | |
| SNP | (p-values) | | Model (p-values | |
| IL4_1158 | 0.0092 | 0.0050 | 0.0016 | 0.0008 |
| IL4_1916 | 0.0085 | 0.0079 | 0.0105 | 0.0058 |
| IL4_2243 | 0.0050 | 0.0104 | 0.0014 | 0.0015 |
| IL4_3900 | 0.0178 | 0.0318 | 0.0022 | 0.0033 |
| IL4_4135 | 0.0097 | 0.0097 | 0.0019 | 0.0013 |

*The fully adjusted model includes age, gender, smoking, diabetes, BMI, CAD/MI, TC:HDL ratio, lipid medication, and the three significant CRP gene SNPs (CRP_2667, CRP_3872, CRP_6192) with which we previously detected an association for CRP levels.

B. Interaction with CRP SNPs.

Since polymorphisms in the CRP gene have been previously associated with hsCRP levels, we aimed to determine if these SNPs also play a role in this population. We detected a positive association signal for three of the six haplotype tagging SNPs in the CRP gene (CRP_{—}2667, rs1800947, p=0.0006; CRP_{—}3872, rs1205, p=0.0109; and CRP_{—}6192, rs3093075, p=0.0169; CRP_{—}969, CRP_{—}1440, CRP_{—}1919, n.s.).

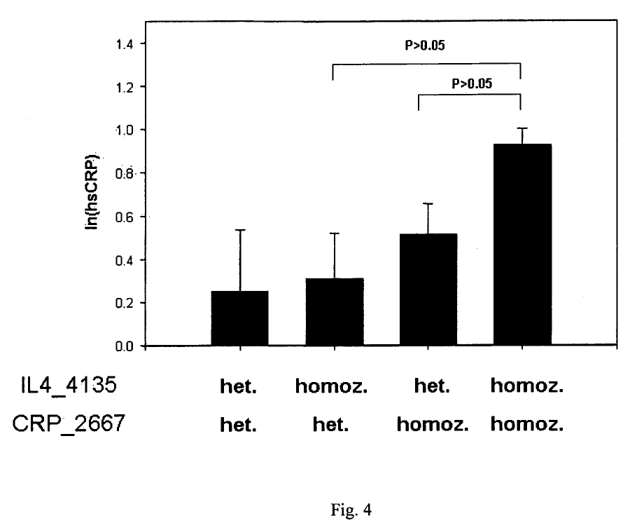

Consequently, we next examined a possible interaction between these three CRP SNPs and the newly identified significant SNPs in the IL4-IL13 intergenic region. Using a multivariate extension of the score-based FBAT statistic, as incorporated in the software package PBAT (Lange C, et al., Am J Hum Genet 2004;74(2):367-9), we identified in our full family set a significant interaction between the intergenic region IL4_{—}4135 SNP and the CRP gene SNPs 2667 and 6192 (p-value for FBAT-I=0.0318; adjusted for age, gender, diabetes, smoking, BMI). In addition, we examined mean InCRP levels by genotype in unrelated affected siblings from our family set (FIG. 4). Overall, we detected a significant difference between the four groups (One Way ANOVA, p=0.007). Individuals homozygous for the common allele of both the CRP_{—}2667 and IL4_{—}4135 SNPs have the highest mean level of InCRP while those who are compound heterozygotes have the lowest levels. The minor/rare allele of IL4_{—}4135 reduces mean InCRP levels by approximately 45% in homozygotes of CRP_{—}2667 compared to individuals homozygous for both SNPs (p<0.05). Furthermore, being heterozygote for CRP_{—}2667 and homozygous for IL4_{—}4135 reduces InCRP values 66% compared to those homozygous for both SNPs (p<0.05) (FIG. 4).

C. Conditional Linkage Analysis.

The next logical step was to determine if these SNPs could in fact account for our initial linkage and could therefore explain the underlying genetic variance. We incorporated all significant SNPs into our original linkage model and then re-evaluated the evidence for linkage, as a model incorporating the causal marker(s) should significantly reduce the initial LOD score. This included also the SNPs in the CRP gene given the combined effect between IL_{—}4 and CRP polymorphisms. The largest reduction in the initial LOD score of 2.24 was achieved with the addition of two of the IL4-IL13 SNPs, IL4_{—}1916 and IL4-4135, and the three CRP SNPs to the conditional linkage model, resulting in a LOD score of 0.68 (FIG. 3: conditional linkage LOD scores in solid line compared to initial plot).

D. In silico and In vitro Functional Analyses.

Prediction of transcription factor binding sites: Since these significant SNPs lie in an intergenic region previously associated with immune regulatory function mediated by various transcription factors, we used transcription factor binding site (TFBS) prediction analysis, as incorporated into Matlnspector (Cartharius K, et al., Bioinformatics 2005;21(13):2933-42), to test whether these SNPs reside within TFBS. Based on this analysis, IL4_{—}1916 lies within two overlapping predicted binding sites, an NF-KappaB p65 site and an Ikaros-1 site. The crucial role of NF-kappaB in inflammation, immune response, cell proliferation, and differentiation is well-established (Siebenlist U, et al., Nat Rev Immunol 2005;5(6):435-45) and Ikaros proteins are critical factors in T lymphocyte development (Georgopoulos K. Nat Rev Immunol 2002;2(3):162-74). IL4_{—}4135 resides within a predicted RBP-Jkappa/CBF-1 site and RBP-Jkappa has recently been shown to regulate IL4 gene transcription as part of the Notch signaling pathway (Amsen D, et al., Cell 2004;1 17(4):515-26). The other SNPS in the haplotype block which also showed a positive association signal did not fall into predicted TFBS. Therefore, we performed the subsequent functional analysis only with these two most likely SNPs.

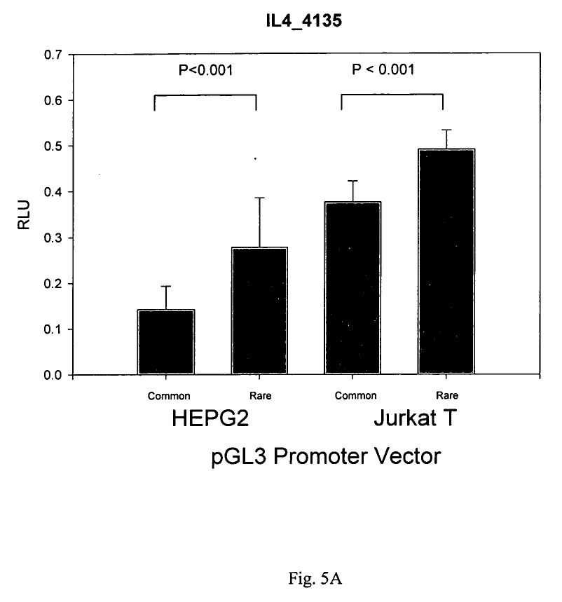

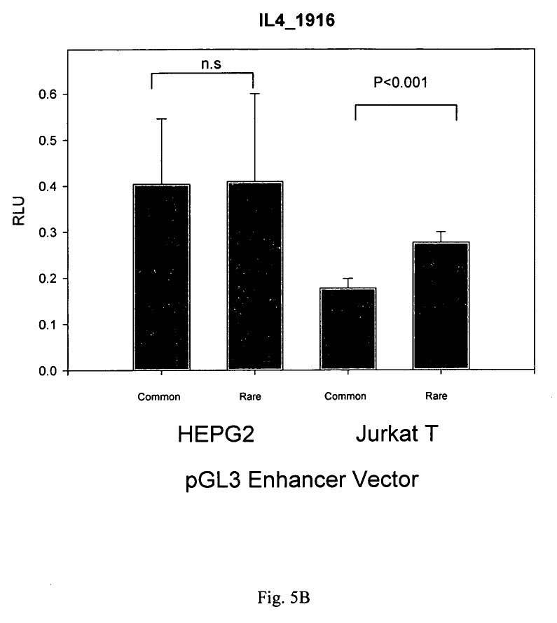

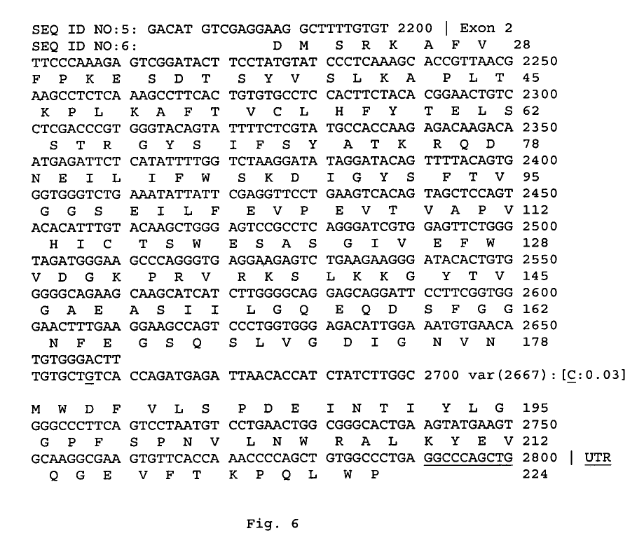

Molecular analysis of SNPs: In order to complement the computational prediction for TFBS, we employed reporter-gene luciferase assays to determine the allele-specific functional relevance of the SNPs via transient transfection assays in both HepG2 and JurkatT- cells. Each of the two alleles for the two significant SNPs (IL4_{—}1916 and IL4_{—}4315) was cloned into a pGL3 luciferase-reporter plasmid. Consistent with the results of our previous analyses, allele-specific differences in reporter gene expression were demonstrated for both markers. The minor/rare allele of IL4_{—}4135 showed a significantly higher luciferase activity in an SV40 promoter vector in comparison to the common allele in HepG2, as well as Jurkat T-cells (FIG. 5A). The minor/rare allele of IL4_{—}1916 also showed an increased luciferase activity using an enhancer vector in Jurkat T-cells, while we did not detect a difference in HepG2 cells (FIG. 5B).

E. Clinical Relevance of Identified SNPs—Time to first MI Analysis.

Given that individuals with increased CRP levels may have an accelerated time to symptom onset and cardiovascular events, we tested the effect of these SNPs on time to first MI in our family set. Using Cox-Proportional Hazards modeling employing a frailty model to adjust for familial correlation, IL4_{—}1916 was shown to significantly predict the time to first MI independent of traditional risk factors, including gender, hyperlipidemia, diabetes and smoking (p=0.018). The minor/rare allele of IL4_{—}1916 exerts a protective effect, conferring a significant 28% reduction in the hazard ratio for MI (HR=0.716, 95% C.I. 0.544-0.944) after adjusting for significant risk factors. Individuals carrying the minor/rare allele of IL4_{—}1916 have a mean age at first MI of 59 years, while those with the common allele have a significantly younger mean age at first MI of 56 years (p=0.017).

Although the invention has been described in connection with specific examples, it is understood that the invention is not limited to such specific examples but encompasses all such modifications and variations apparent to a skilled artisan that fall within the scope of the appended claims.

We claim:

1. A method of screening a human subject for propensity to develop a cardiovascular disease comprising the steps of:
(a) determining the status of a marker selected from single nucleotide polymorphism (SNP) marker IL4_{—}4135 or another SNP marker in linkage disequilibrium with IL4_{—}4135 in the genome of the human subject; and
(b) correlating the result from step (a) to the subject's propensity for developing a cardiovascular disease wherein subjects who carry the minor allele of IL4_{—}4135 are less likely to develop a cardiovascular disease than subjects who do not carry the minor allele of IL4_{—}4135.

2. The method of claim 1, wherein the cardiovascular disease is selected from stroke, coronary artery disease, end stage renal disease, and peripheral artery disease.

3. The method of claim 2, wherein the coronary artery disease is myocardial infarction.

4. A method of correlating a human subject's serum or plasma C-reactive protein (CRP) level to the subject's genetic composition comprising the steps of:
(a) determining the status of a marker selected from single nucleotide polymorphism (SNP) marker IL4_{—}4135 or another SNP marker in linkage disequilibrium with IL4_{—}4135 in the genome of the human subject; and
(b) correlating the result from step (a) to the subject's serum or plasma CRP level wherein subjects who carry the minor allele of IL4_{—}4135 have a lower average serum or plasma CRP level than subjects who do not carry the minor allele of IL4_{—}4135.

5. A method of screening a human subject for propensity to develop a cardiovascular disease comprising the steps of:
(a) genotyping the genome of the human subject for SNP marker IL4_{—}4135 or another SNP marker that is in linkage disequilibrium with IL4_{—}4135;
(b) genotyping the genome of the human subject for SNP marker CRP_{—}2667 or another SNP marker that is in linkage disequilibrium with CRP_{—}2667; and
(c) correlating the results from steps (a) and (b) to the subject's propensity for developing a cardiovascular disease wherein individuals who are homozygous for the common allele of both IL4_{—}4135 and CRP_{—}2667 are more likely to develop a cardiovascular disease than individuals who are heterozygous for one and homozygous for the other of IL4_{—}4135 and CRP_{—}2667, who are in turn more likely to develop a cardiovascular disease than individuals who are homozygous for the minor allele of both IL4_{—}4135 and CRP_{—}2667.

6. The method of claim 5, wherein the cardiovascular disease is selected from stroke, coronary artery disease, end stage renal disease, and peripheral artery disease.

7. The method of claim 6, wherein the coronary artery disease is myocardial infarction.

8. A method of correlating a human subject's serum or plasma C-reactive protein (CRP) level to the subject's genetic composition comprising the steps of:
(a) genotyping the genome of the human subject for SNP marker IL4_{—}4135 or another SNP marker that is in linkage disequilibrium with IL4_{—}4135;
(b) genotyping the genome of the human subject for SNP marker CRP_{—}2667 or another SNP marker that is in linkage disequilibrium with CRP_{—}2667; and
(c) correlating the results from steps (a) and (b) to the subject's serum or plasma CRP level wherein individuals who are homozygous for the common allele of both IL4_{—}4135 and CRP_{—}2667 have a higher average CRP level than individuals who are heterozygous for one and homozygous for the other of IL4_{—}4135 and CRP_{—}2667, who are in turn have a higher average CRP level than individuals who are homozygous for the minor allele of both IL4_{—}4135 and CRP_{—}2667.

9. A method of screening a human subject for predicting time to first myocardial infarction comprising the steps of:
(a) determining the status of a marker selected from SNP marker IL4_{—}1916 or another SNP marker in linkage disequilibrium with IL4_{—}1916 in the genome of the human subject; and
(b) correlating the result from step (a) to time to first myocardial infarction wherein subjects who carry the minor allele of IL4_{—}1916 are likely to have myocardial infarction for the first time at an older age than subjects who do not carry the minor allele of IL4_{—}1916.
